(19) United States
(12) Patent Application Publication
(10) Pub. No.: US 2024/0165195 A1
(43) Pub. Date: May 23, 2024

(54) USE OF ALPHA-V-INTEGRIN (CD51) INHIBITORS FOR THE TREATMENT OF CARDIAC FIBROSIS

(71) Applicants: INSERM (Institut National de la Santé et de la Recherche Médicale), Paris (FR); Université Paris Cité, Paris (FR); Assistance Publique-Hôpitaux de Paris (APHP), Paris (FR)

(72) Inventor: Jean-Sébastien HULOT, Paris (FR)

(21) Appl. No.: 18/460,962

(22) Filed: Sep. 5, 2023

Related U.S. Application Data
(63) Continuation-in-part of application No. 17/282,599, filed on Apr. 2, 2021.

(51) Int. Cl. A61K 38/12; A61P 9/04; C07K 16/28

(52) U.S. Cl. CPC A61K 38/12; A61P 9/04; C07K 16/2848

(57) ABSTRACT

Activated cardiac fibroblasts are essential for the production of extracellular matrix proteins that accumulate during cardiac fibrosis, and PW1⁺ cardiac adult stem cells were recently proposed as a cellular source of fibroblasts in the ischemic hearts. Here the inventors identify αV-integrin (or CD51) as an essential regulator of PW1⁺ cardiac adult stem cells fibrogenic behavior. Inhibition of αV-integrin reduce the profibrotic gene expression profile and the ability to differentiate into fibroblasts of cardiac PW1+ cells. The pharmacological blockade of αV-containing integrins improved cardiac function and survival after MI by reducing infarct size and attenuating the extension of reactive cardiac fibrosis. Notably, the total cardiac fibrotic area as well as interstitial fibrosis in the remote myocardial area are significantly reduced after pharmacological blockade of αV-containing integrins. These data identify a new mechanism that regulates cardiac fibrosis in response to an ischemic injury and suggest that pharmacological targeting of αV-integrin may provide clinical benefit in the treatment of cardiac fibrosis.

### CROSS-REFERENCE TO RELATED APPLICATIONS

This application is a continuation-in-part application of U.S. Ser. No. 17/282,589 filed Apr. 2, 2021 which was a Rule 371 filing from PCT/IB2018/001394 filed Oct. 9, 2018. This application also claims priority to U.S. Ser. No. 63/409,245 filed Sep. 23, 2022, the contents of which are herein incorporated by reference.

### SEQUENCE LISTING

This document incorporates by reference an electronic sequence listing XML file, which was electronically submitted along with this document. The XML file is named 2023-09-05_11450676US2_seqlisting.xml, is 3044 bytes, and was created on Aug. 8, 2023.

### FIELD OF THE INVENTION

The present invention relates to use of αV-integrin (CD51) inhibitors for the treatment of cardiac fibrosis.

### BACKGROUND OF THE INVENTION

Heart failure (HF) remains a leading cause of mortality and hospitalizations worldwide and represents a heavy health care financial burden¹⁻⁶. Current pharmacological treatments limit the peripheral consequences of cardiac dysfunction but there is a limited number of therapeutics that impact the primary adverse cardiac remodeling at the myocardial level. While the etiology of HF is diverse, HF is typically associated with a range of physiological and morphological changes including fibrosis of the myocardium⁷⁻⁹. Indeed, cardiac fibrosis is characterized by the excessive production and deposition of extracellular matrix (ECM) proteins into the myocardium which leads to normal tissue architecture disruption, reduced tissue compliance, mechanical and electrical dysfunction and which eventually accelerates the progression to HF^{10,11}. However, as the mechanisms contributing to cardiac fibrosis are incompletely understood, there is currently no effective anti-fibrotic therapeutic strategy that could complement the current therapies for HF^{9,11,12}.

Activated cardiac fibroblasts are essential for the production of ECM proteins that accumulate during cardiac fibrosis, however, recent studies have established that cardiac fibroblasts represent a very heterogeneous cell population¹⁰⁻¹⁴. The exact nature of activated fibroblasts and consequently the sources of cardiac fibrosis remain poorly understood^{9,12}. Different mechanisms have been reported including proliferation and activation of resident fibroblasts¹⁵, transformation of endothelial and/or epicardial cells after injury through endothelial-mesenchymal transition and epithelial-mesenchymal transition respectively^{16,17}, migration of hematopoietic bone marrow-derived cells and perivascular cells¹⁸. Another model proposes that tissue resident progenitor populations activate in response to stress and become a major cellular source of organ fibrosis, including in the heart. In a recent study, we have identified a novel population of cardiac cells that reside in the myocardium and have a fibrogenic behavior in response to cardiac ischemic injury¹⁹. This population was identified based upon the expression of the pan-stem cell marker, Pw1/Peg3 (referred hereafter as PW1)^{20,21}, using a transgenic Pw1-beta galactosidase reporter mouse model. Taking advantage of the strong and produrant expression of the β-gal reporter enzyme, we found that a significant proportion (˜22%) of fibroblasts were derived from PW1 expressing cells in ischemic hearts. therefore suggesting that cardiac PW1⁺ cells can contribute to cardiac fibrosis by directly giving rise to fibroblasts thus representing a source of additional ECM.

### SUMMARY OF THE INVENTION

The present invention relates to use of αV-integrin (CD51) inhibitors for the treatment of cardiac fibrosis. In particular, the present invention is defined by the claims.

### DETAILED DESCRIPTION OF THE INVENTION

Activated cardiac fibroblasts are essential for the production of extracellular matrix proteins that accumulate during cardiac fibrosis, and PW1⁺ cardiac adult stem cells were recently proposed as a cellular source of fibroblasts in the ischemic hearts. Here the inventors identify αV-integrin (or CD51) as an essential regulator of PW1⁺ cardiac adult stem cells fibrogenic behavior. Using a combination of transcriptomic and proteomic approaches, they identified the presence of αV-integrin in the plasma membrane of cardiac PW1+ cells. Expression analyses revealed αV-integrin expression as highly sensitive and specific to cardiac PW1+ cells. More than 93% of FACS-isolated cardiac PW1⁺ cells express CD51 and reciprocally more than 85% of PW1+ cells are recovered among the FACS-isolated CD51⁺ cardiac cells. Inhibition of αV-integrin reduce the profibrotic gene expression profile and the ability to differentiate into fibroblasts of cardiac PW1+ cells. Cardiac PW1+ cells showed a predominant expression of αVβ1 complex, a putative key mediator of organ fibrosis through TGF-beta activation. Consequently, pharmacological blockade of αV-containing integrins improved cardiac function and survival after MI by reducing infarct size and attenuating the extension of reactive cardiac fibrosis. Notably, the total cardiac fibrotic area as well as interstitial fibrosis in the remote myocardial area are significantly reduced after pharmacological blockade of αV-containing integrins. These data identify a new mechanism that regulates cardiac fibrosis in response to an ischemic injury and suggest that pharmacological targeting of αV-integrin may provide clinical benefit in the treatment of cardiac fibrosis.

Accordingly, the first object of the present invention relates to a method of treating cardiac fibrosis in a patient in need thereof comprising administering to the patient a therapeutically effective amount of a αV-integrin inhibitor.

As used herein, the term “cardiac fibrosis” has its general meaning in the art and refers to a condition characterized by the excessive production and deposition of extracellular matrix (ECM) proteins into the myocardium which leads to normal tissue architecture disruption, reduced tissue compliance, mechanical and electrical dysfunction. Cardiac fibrosis arises from aging, exposure to certain drugs, or in response to various heart diseases, such as myocardial infarction and hypertension. Following acute myocardial infarction, sudden loss of a large number of cardiomyocytes triggers an inflammatory reaction, ultimately leading to replacement of dead myocardium with a collagen-based scar. Several other pathophysiologic conditions induce more insidious interstitial and perivascular deposition of collagen, in the absence of completed infarction. Aging is associated with progressive fibrosis that may contribute to the development of diastolic heart failure in elderly patients. Pressure overload, induced by hypertension or aortic stenosis, results in extensive cardiac fibrosis that is initially associated with increased stiffness and diastolic dysfunction. Volume overload due to valvular regurgitant lesions may also result in cardiac fibrosis. Hypertrophic cardiomyopathy and post-viral dilated cardiomyopathy are also often associated with the development of significant cardiac fibrosis. Moreover, a variety of toxic insults (such as alcohol or anthracyclines) and metabolic disturbances (such as diabetes and obesity) induce cardiac fibrosis.

In particular, the αV-integrin inhibitor of the present invention is particularly suitable for limiting the development of reactive interstitial fibrosis in the viable myocardium. More particularly, the αV-integrin inhibitor of the present invention is suitable for improving cardiac function in a subject suffering from cardiac fibrosis. As used herein, the term “cardiac function” refers to the function of the heart, including global and regional functions of the heart. The term “global” cardiac function as used herein refers to function of the heart as a whole. Such function can be measured by, for example, stroke volume, ejection fraction, cardiac output, cardiac contractility, etc. The term “regional cardiac function” refers to the function of a portion or region of the heart. Such regional function can be measured, for example, by wall thickening, wall motion, myocardial mass, segmental shortening, ventricular remodeling, new muscle formation, the percentage of cardiac cell proliferation and programmed cell death, angiogenesis and the size of fibrous and infarct tissue. More particularly, the αV-integrin inhibitor of the present invention is suitable for improving survival of a subject suffering from cardiac fibrosis.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “αV-integrin” or “CD51” has its general meaning in the art and refers to that protein that in humans is encoded by the ITGAV gene. An exemplary human amino acid sequence of CD51 is represented by SEQ ID NO; 1. Integrins are heterodimeric integral membrane proteins composed of an alpha chain and a beta chain. αV undergoes post-translational cleavage to yield disulfide-linked heavy and light chains, that combine with multiple integrin beta chains to form different integrins. Among the known associating beta chains (beta chains 1,3,5,6, and 8; ‘ITGB1’, ‘ITGB3’, ‘ITGB5’, ‘ITGB6’, and ‘ITGB8’), each can interact with extracellular matrix ligands.

>sp|P06756|ITAV_HUMAN Integrin alpha-V OS =
Homo sapiens OX = 9606 GN = ITGAV PE = 1 SV = 2
SEQ ID NO: 1
MAFPPRRRLRLGPRGLPLLLSGLLLPLCRAFNLDVDSPAEYSGPEGSYFG
FAVDFFVPSASSRMFLLVGAPKANTTQPGIVEGGQVLKCDWSSTRRCQPI
EFDATGNRDYAKDDPLEFKSHQWFGASVRSKQDKILACAPLYHWRTEMKQ
EREPVGTCFLQDGTKTVEYAPCRSQDIDADGQGFCQGGFSIDFTKADRVL
LGGPGSFYWQGQLISDQVAEIVSKYDPNVYSIKYNNQLATRTAQAIFDDS
YLGYSVAVGDFNGDGIDDFVSGVPRAARTLGMVYIYDGKNMSSLYNFTGE
QMAAYFGFSVAATDINGDDYADVFIGAPLFMDRGSDGKLQEVGQVSVSLQ
RASGDFQTTKLNGFEVFARFGSAIAPLGDLDQDGFNDIAIAAPYGGEDKK
GIVYIFNGRSTGLNAVPSQILEGQWAARSMPPSFGYSMKGATDIDKNGYP
DLIVGAFGVDRAILYRARPVITVNAGLEVYPSILNQDNKTCSLPGTALKV
SCFNVRFCLKADGKGVLPRKLNFQVELLLDKLKQKGAIRRALFLYSRSPS
HSKNMTISRGGLMQCEELIAYLRDESEFRDKLTPITIFMEYRLDYRTAAD
TTGLQPILNQFTPANISRQAHILLDCGEDNVCKPKLEVSVDSDQKKIYIG
DDNPLTLIVKAQNQGEGAYEAELIVSIPLQADFIGVVRNNEALARLSCAF
KTENQTRQVVCDLGNPMKAGTQLLAGLRFSVHQQSEMDTSVKFDLQIQSS
NLFDKVSPVVSHKVDLAVLAAVEIRGVSSPDHVFLPIPNWEHKENPETEE
DVGPVVQHIYELRNNGPSSFSKAMLHLQWPYKYNNNTLLYILHYDIDGPM
NCTSDMEINPLRIKISSLQTTEKNDTVAGQGERDHLITKRDLALSEGDIH
TLGCGVAQCLKIVCQVGRLDRGKSAILYVKSLLWTETFMNKENQNHSYSL
KSSASFNVIEFPYKNLPIEDITNSTLVTTNVTWGIQPAPMPVPVWVIILA
VLAGLLLLAVLVFVMYRMGFFKRVRPPQEEQEREQLQPHENGEGNSET

As used herein the “αV-integrin inhibitor” refers to refers to any compound natural or not which is capable of inhibiting the activity or expression of αV-integrin. The term encompasses any antagonist that is currently known in the art or that will be identified in the future, and includes any chemical entity that, upon administration to a patient, results in inhibition or down-regulation of a biological activity associated with αV-integrin. The inhibitor can thus inhibit the expression or activity of αV-integrin, modulate or block the αV-integrin signaling pathway and/or block the binding of αV-integrin to a binding partner. The term also encompasses inhibitor of expression.

A significant number of αV-integrin antagonists (Goodman et al, Trends in Pharmacological Sciences, 2012, 33, 405; Tucker, Gordon C. “Alpha v integrin inhibitors and cancer therapy.” Current opinion in investigational drugs (London, England: 2000) 4.6 (2003): 722-731; Hatley, Richard J D, et al. “An αv-RGD Integrin Inhibitor Toolbox: Drug Discovery Insight, Challenges and Opportunities.” Angewandte Chemie International Edition 57.13 (2018): 3298-3321.) have been disclosed in the literature including inhibitory antibodies, peptides and small molecules.

In some embodiments, the αV-integrin inhibitor of the present invention is an antibody, more particularly an antibody having specificity for αV-integrin.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161 ; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001 ; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388.

In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. Fully human monoclonal antibodies also can be prepared by immunizing mice transgenic for large portions of human immunoglobulin heavy and light chain loci. Sec, e.g., U.S. Pat. Nos. 5,591,669, 5,598,369, 5,545,806, 5,545,807, 6,150,584, and references cited therein, the contents of which are incorporated herein by reference.

In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “nanobody®”.

In some embodiments, the antibody comprises human heavy chain constant regions sequences but will not induce antibody dependent cellular cytotoxicity (ADCC). In some embodiments, the antibody of the present invention does not comprise an Fc domain capable of substantially binding to a FcgRIIIA (CD16) polypeptide. In some embodiments, the antibody of the present invention lacks an Fc domain (e.g. lacks a CH2 and/or CH3 domain) or comprises an Fc domain of IgG2 or IgG4 isotype. In some embodiments, the antibody of the present invention consists of or comprises a Fab, Fab′, Fab′-SH, F (ab′) 2, Fv, a diabody, single-chain antibody fragment, or a multispecific antibody comprising multiple different antibody fragments. In some embodiments, the antibody of the present invention is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. Nos. 6,194,551 by ldusogie et al.

Antibodies having specificity for αV-integrin are well known in the art and typically include intetumumab and abituzumab. Intetumumab is a pan alpha-v human monoclonal antibody that recognizes alpha-v beta-1, alpha-v beta-3, alpha-v beta-5, and alpha-v beta-6 integrins. Intetumumab competitively binds to and blocks both alpha-v beta-3 and alpha-v beta-5 integrins Abituzumab is a humanized monoclonal IgG2 antibody that specifically targets all αv integrins (Mitjans F, et al. J Cell Sci 1995; 108:2825-38; Monnier Y, et al. Cancer Res 2008:68; 7323-31).

In some embodiments, the αV-integrin inhibitor is Cilengitide. Cilengitide is a cyclic peptide antagonist that inhibits both αvβ1, αvβ3 and αvβ5. The IUPAC name of Cilengitide is 2-[(2S,5R,8S, 11S)-5-benzyl-11-{3-[(diaminomethylidene)amino]propyl}-7-methyl-3,6,9,12,15-pentaoxo-8-(propan-2-yl)-1,4,7,10,13-pentaazacyclopentadecan-2-yl]acetic acid.

In some embodiments, the αV-integrin inhibitor is an inhibitor of αV-integrin expression respectively. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of αV-integrin mRNA by binding thereto and thus preventing protein translation or increasing mRNA degradation, thus decreasing the level of αV-integrin, and thus activity, in a cell. For example, antisense oligonucleotides of at least about 15 bases and complementary to unique regions of the mRNA transcript sequence encoding αV-integrin can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. αV-integrin gene expression can be reduced by contacting a patient or cell with a small double stranded RNA (dsRNA), or a vector or construct causing the production of a small double stranded RNA, such that αV-integrin gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing αV-integrin. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor of expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

By a “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically the active ingredient of the present invention (e.g. αV-integrin inhibitor) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The invention will be further illustrated by the following figures and examples. However, these examples and figures should not be interpreted in any way as limiting the scope of the present invention.

### FIGURES

FIGS. 1A-E. Cilengitide reduces cardiac fibrosis and improves cardiac function after MI. (A) Experimental design. Mice were pre-treated for 7 days with cilengitide or vehicle (n=16 per groups) before surgery and complete LAD ligation as an MI model (defined as Day 0). The mice were then followed and treated with cilengitide or vehicle for seven additional days. Final non-invasive and invasive evaluations were performed 7 days after surgery. (B) Survival curves in cilengitide vs vehicle treated mice in the seven days after MI. *p<0.05, log-rank test. (C) Left ventricular ejection fractions in cilengitide vs vehicle treated mice immediately before surgery and 7 days later. **p<0.01. ****p<0.0001. (D) Typical masson trichome staining in vehicle and cilengitide treated animals and quantification of infarct size between both groups, ***p<0.001, n=4 animal per groups. (E) Typical picrosirius red staining in vehicle and cilengitide treated animals and quantification of interstitial fibrosis in remote areas between both groups, ****p<0.0001, n=4 animal per groups.

FIGS. 2A-C. Cilengitide reduces the profibrotic activation of cardiac PW1⁺ cells (A) Quantitative PCR evaluation of acta2, mmp2, tgfrb1 and colla1 expression in isolated cardiac PW1⁺ cells cultured in presence of escalating concentrations of cilengitide (0 for vehicle only, 300 nM, 1000 nM). Data are expressed as means±SEM (n=4 in duplicate). *p<0.05, **p<0.01 and ***p<0.001 for Kruskal-Wallis test between all groups; #p<0.05, ##p<0.01 for Dunn's comparison with vehicle treated cells. (B) Quantification of β-gal+ cells within the vimentin-positive cells in vehicle and cilengitide post-MI heart sections, n=30 fields from 3 animals per groups, *p<0.05. (C). Quantification of β-gal+ and vimentin-negative cells within the total number of cells in vehicle and cilengitide post-MI heart sections, n=30 fields from 3 animals per groups. ***p<0.001.

FIGS. 3A-J. HHF model induces cardiac fibrosis with transition to heart failure. (A) Experimental design. (B) Statistical analysis of the cardiac mass between models, as measured by heart weight on body weight ratio. (C) Immunofluorescent staining of plasma membrane by WGA and endothelial cells by CD31 marking on FFPE cardiac cross-sections and (D) corresponding statistical analysis of cardiomyocyte diameter. (E) Picrosirius staining of collagen fibers on FFPE cardiac cross-sections and (F) corresponding statistical analysis of the amount of interstitial fibrosis. (G) Statistical analysis of Lung Weight on Body Weight ratio. (H) Statistical analysis of the Left Ventricular Ejection Fraction, (I) Left Atria size and (J) E/E′ ratio assessed via cardiac echography. Mean±SD, *p<0.05, **p<0.01. ***p<0.001, ****p<0.0001.

FIGS. 4A-G. CD51 expression is increased in HHF mice. (A) Western-Blot showing CD51 protein expression in CCH and HHF mice, with vinculin as loading control and (B) corresponding statistical analysis. (C) Statistical analysis of the total number of CD51+ cells found in the myocardium in the different models, assessed by flow cytometry, per million cells analyzed. (D) CD51 expression in non-hematopoietic cardiac cells and (E) statistical analysis of the quantity of non-hematopoietic CD51+ cells found in the myocardium, per million cells. (F) CD51 expression in hematopoietic cardiac cells and (G) Statistical analysis of the quantity of hematopoietic CD51+ cells found in the myocardium, per million cells.

FIGS. 5A-I. Cilengitide reduces cardiac fibrosis and transition to heart failure. (A) Experimental design. (B) Statistical analysis of the cardiac mass, as measured by heart weight on body weight ratio. (C) Statistical analysis of the cardiomyocyte size. (D) Picrosirius Staining of collagen fibers on FFPE cardiac cross-sections and (E) corresponding statistical analysis of interstitial fibrosis. (F) Statistical analysis of the Lung Weight on Body Weight ratio, (G) Left Ventricular Ejection Fraction. (H) Left Atria size and (I) E/E′ ratio, assessed via cardiac echography. Mean±SD, *p<0.05. **p<0.01. ***p<0.001. ****p<0.0001.

FIGS. 6A-K. CD51 identifies inflammatory monocyte and monocyte-derived macrophages. (A) Flow Cytometry analysis with sequential narrowing of cells with high percentage of CD51 expression. (B) Statistical analysis of the quantity of F4/80+CD64+cardiac macrophages in the different models, assessed by flow cytometry, per million cell analyzed. (C) Flow cytometry representation of the expression of CCR2 in F4/80+CD64+macrophages. (D) Flow cytometry representation of the expression of CD51 in the CCR2+macrophage population and (E) corresponding statistical analysis. (F) Statistical analysis of the quantity of cardiac Ly6C+monocytes in the different models, assessed by flow cytometry, per million cells analyzed. (G) Flow cytometry representation of Ly6Chighand Ly6Cmidcardiacmonocytes, versus CD51 expression. (H) Flow cytometry representation of the expression of CD51 in the Ly6Chighpopulation and (I) corresponding statistical analysis. (J) Flow cytometry representation of the expression of CD51 in the Ly6Cmidpopulation and (K) corresponding statistical analysis.

FIGS. 7A-G. Cilengitide reduces inflammatory cells' number and pro-inflammatory cytokines' secretion. (A) Statistical analysis of the quantity of cardiac CCR2+pro-inflammatory macrophages, assessed by flow cytometry, per million cells. (B) Statistical analysis of the quantity of cardiac Ly6C+monocytes, assessed by flow cytometry, per million cells. (C) Statistical analysis of the quantity of cardiac Ly6Chighmonocytes, assessed by flow cytometry, per million cells. (D) Statistical analysis of the quantity of cardiac Ly6Cmidmonocytes, assessed by flow cytometry, per million cells. (E) Experimental design of the BMDMs culture with different matrix stiffness on a vitronectin coating. (F) Statistical analysis of IL-1 beta secretion by the BMDMs and (G) Statistical analysis of IL-6 secretion by the BMDMs, measured by immunoassay. Mean±SD. *p<0.05, **p<0.01, ***p<0.001, ****p<0.0001.

### EXAMPLE 1

### Methods

All procedures and animal care were approved by our institutional research committee and conformed the animal care guideline in Directive 2010/63/EU European Parliament.

### Myocardial Infarction by LAD Ligation

Male 8 weeks-old C57BL/6 or PW1-reporter (PW1^{nLacZ}) mice were anesthetized in an induction chamber with 2% isoflurane mixed with 1.0 L/min 100% O₂ and placed on a supine position on a heating pad to maintain body temperature. The mice were intubated with endotracheal tube and then connected to a rodent ventilator (180 breaths/min and a tidal volume of 200 μl). During surgical procedure anesthesia is maintained at 1.5-2% isoflurane with O₂. The chest was accessed from the left side through the intercostal space and the pericardium incised. The left anterior descending coronary artery (LAD) was exposed and encircled with a 8.0 prolene suture at the apex of the left ventricle. The suture was briefly snared to confirm the ligation by blanching the arterial region. Mice were analyzed 7 days after LAD permanent ligation.

Male C57BL/6J mice (Janvier labs) or PW1-reporter (PW1^{nLacZ}) mice received Cilengitide at 10 mg/kg/day (Adooq bioscience, ref: A12372) or vehicle i.p. for 14 days. LAD surgery was performed 7 days after starting cilengitide or vehicle administration. Mice were divided in 4 groups: MI-CIL (Cilengitide), MI-Veh (vehicle), Ctrl-CIL and Ctrl-Veh. At Day 14, animals were euthanatized and hearts were carefully collected, incubated for 2 h at 4° C. in 4% Formaldehyde solution (Ted Pella) then 24 h in 30% sucrose/PBS solution before OCT embedding and frozen in liquid nitrogen vapours. Echocardiographic measurements were taken at days 0 and 7 to assess cardiac function.

### Cell Isolation and Fluorescence-Activated Cell Sorting

Animals were injected with heparin (100 UI/25 g) before euthanasia. The right atrium was carefully nicked with surgical scissors and the heart was perfused with 10 mL of PBS (Gibco) through the right ventricle to remove the blood. After collecting the heart, the atria were trimmed away and the ventricles were finely sliced (0.5-1 mm³). Cardiac cells were dissociated at 37ºC for 30 min by enzymatic digestion with 480U/mL collagenase II (Worthington) in DPBS (GIBCO). The digestion was stopped with DMEM (Life Technologies) supplemented into 10% FBS (Sigma) and 1% penicillin/streptomycin (P/S; Life Technologies) and the cell suspension was filtered through 100 μm Falcon cell strainer before centrifugation at 416 g for 10 min at 4° C. The cell pellet was re-suspended in 500 μL of Red Blood Cell Lysing (RBCL) Buffer to lyse red blood cells at RT for 30 sec. DPBS was added before 10 min centrifugation in same conditions as above. The cell pellet was then re-suspended in 300 μL of HBSS (Gibco) containing 1% FBS (HBSS-1% FBS) to perform immunostaining for 45 min on ice in dark. The list of antibodies used is reported in supplemental table 1. After incubation, HBSS-1% FBS was added and cells centrifuged at 416 g for 10 min at 4° C. to perform PW1⁺ cells staining. For this, 300 μL of HBSS-1% FBS containing 60 μM of 5-Dodecanoylaminofluorescein Di-β-D-Galactopyranoside (C₁₂FDG, ThermoFisher Scientific) a substrate for β-gal detection by flow cytometry was added on cells then incubated in dark at 37° C. for 1 h under constant agitation. Reaction was stopped by the addition of HBSS-1% FBS. After cell centrifugation, the pellet was re-suspended in 200 μL of HBSS-1% FBS, the viability dye propidium iodide (Sigma) was added and the samples were filtered through a 50 μm Filcon (BD bioscience) just before the analysis.

Viability threshold was determined by comparing conditions with and without viability dye. All immunostained populations were obtained by comparing signals from unstained cells and Fluorescence minus one conditions. B-Gal activity threshold was obtained by comparing β-Gal negative and positive cells.

### RNA-Seq

300 ng of total RNA extracted from freshly isolated cells were used to perform library preparation with SureSelect Strand-Specific RNA kit (Agilent), according to the manufacturer's instructions. The resulting library was quality checked and quantified by peak integration on Bioanalyzer High sensitivity DNA labchip (Agilent). A pool of equal quantity of 12 purified libraries was done, each library being tagged with a different index. miRNAs pool libraries were finally sequenced on Illumina Hiseq 1500 instrument using a rapid flowcell. The pool was loading on 2 lanes of the flowcell. A paired End sequencing of 2×100 bp was performed.

After discarding reads that did not pass the Illumina filters and trimming sequenced bases with low quality (q<28) using the Cutadapt program²⁴, we restricted our downstream analyses to reads with length greater than 90 bp. Selected reads were mapped to a murine reference transcriptome that was generated by the RSEM package²⁵ from the full mouse reference genome and the gtf transcript annotations file from ENSEMBL²⁶. Alignment and estimation of transcripts abundance in each of the 12 processed samples were performed using the RSEM program. Transcripts with abundance counts higher than 10 in more than 2 samples (N=36,948) were considered as expressed and kept for further analysis. Abundances of transcripts assigned to the same gene were combined together leading to the profiling of 16,403 gene expressions. A principal component analysis was then performed on the 12 expression profiles and revealed that the first three principal components explained ˜84% of the total variability of gene expression profiling. The percentage of variability explained by the first, second and third component were 39.4%, 28.5% and 15.7%, respectively. Analyses were conducted under the R environment (version 3.2.2).

A Galaxy 15.10 instance was locally installed on a server machine. WolfPsort, TMHMM, and SignalP were obtained from CBS prediction servers (cbs.dtu.dk/services/). As NetNEs was not implemented in Galaxy, NLStradamus and PredictNLS were used in replacement. Each population RNASeq was then processed through a pipeline designed to select sequences containing a signal peptide, at least 1 transmembrane segment, no nuclear export signal and a theoretical presence at the plasma membrane. Intentionally loose filters were chosen as putative membranome were manually quality checked for false positive sequences and compared to several databases (Qiagen's Ingenuity Pathway Analysis, Uniprot, Ensembl). Finally, all samples were compared and only sequences found only in PW1+ cells but insensible to myocardial infarction.

### Proteomics

PW1⁺ FACS sorted cells samples were prepared for MS analysis with an adapted protocol as too few cells was recovered to perform the complete protocol. 300 000 PW1⁺ sorted cells were centrifuged at 500 g for 10 min at 4° C. Pellet was lysed in 100 μL of lysis buffer (10 mM HEPES, 1.5 mM MgCl2, 10 mM KCl, 0.5 mM DTT, 1 mM orthovanadate and protein inhibitor cocktail) and incubated for 15 min on ice. A mechanical cell disruption was then performed with 20 up and downs with a dounce potter. Samples were then centrifuged at 4° C. 10 min at 600 g. Supernatant was then centrifuged at 100 000 g for 25 min at 4° C. Pellet was rinsed with 150 μL of rinse buffer and centrifuged in the same conditions as above. The final pellet was resuspended with 150 μL of NH4HCO₃ 50 mM and homogenized. Membranous proteins were analysed on SDS-PAGE gels (4% stacking and a 12% running gel) stained with a MS-compliant silver nitrate staining protocol. All proteins were excised in one large strip which was minced in 1 mm³ cubes just before protein reduction, alkylation (DTT, lodoacetamide) and trypsin digestion (500 ng in AMBIC 50 mM, ACN 5%). Finally, peptides were extracted with 20 μL of ACN 30% FA 0,1%. Mass spectrometry analysis was performed with 4 μL injections on an online LC-MS composed by a nanoHPLC Dionex Ultimate 3000 and a Thermofisher QExactive+with a 2 h gradient. All the proteomic steps were done under the supervision of the proteomic facility from La Pitié-Salpétrière (P3S, UPMC), the LC-MS analysis has been performed on the proteomic facility of Paris-Descartes university (3P5, Paris Descartes).

The software used was X!Tandem (Ver: 2015.04.01.1), using X!Tandem pipeline (Ver: 3.4.3) and the Uniprot reference proteome used was downloaded on Dec. 12, 2016. Standard contaminants were removed. Filter applied: Proteins with at least 1 peptide with e value <0,05; FDR peptide=0.128; FDR protein=0.95. Final protein score Log(e value)<−2. Final results gave 1679 groups with 1831 subgroups.

The protein list obtained from LC-MS analysis has been manually filtered to keep only plasma membrane proteins. This observed PW1⁺ membranome has been confronted with the putative PW1+ membranome. Only proteins found in both lists were treated. Potential candidates were then selected according to literature and antibodies validated for flow cytometry & immunofluorescence staining availability.

### Western Blot Analysis

Proteins from mouse heart were extracted from frozen tissues with Dounce-Potter homogenization into ice-cold RIPA buffer (50 mM Tris pH7.4, 150 mM NaCl, 1% Igepal CA-630, 50 mM deoxycholate, and 0.1% SDS) containing antiproteases (Sigma-Aldrich), serine/threonine and tyrosine protein phosphatase inhibitors (Phosphatase inhibitor Cocktail 2 and 3, Sigma-Aldrich) and 1 mM Na₃ VO₄ or with urea-thiourea buffer (5 M urea, 2 M thiourea, 50 mM DTT , 0.1% [w/v] SDS in PBS pH 7.4). After 1 h incubation at 4° C., the homogenate was centrifuged at 15 300 g for 15 min at 4° C. and the supernatant containing proteins was collected. Protein concentrations for all samples were determined with a Bradford-based method protein assay (Biorad).

After sorting, the heart PW1⁺ cells of 22 mice and the heart PW1⁻ cells of 16 mice were pooled and centrifuged at 500 g for 15 min at 4° C. and lysed in urea-thiourea buffer. Proteins were extracted as describe above.

Cardiac myocytes (CM) and non-cardiomyocyte (non-CM) cells were isolated from the adult mouse heart as previously described with minor modifications (Ackers-Johnson et al, 2016). After cell collection, CM were lysed in RIPA and urea-thiourea buffer for protein extraction as describe above and non-CM were plated in growth medium (DMEM 10% FBS 1% P/S). Proteins were extracted in RIPA and urea-thiourea buffer when cells reached 80% confluence.

Proteins (10-30 μg) were mixed with NuPAGE™ LDS sample buffer, NuPAGE™ Sample Reducing Agent and extraction buffer, incubated at 70° C. for 10 min and then loaded onto a gel lane of a NuPAGE™ Novex® 4-12% Bis-Tris gel (Life Technologies). After 3 h running at 90 V with NuPAGE™ MES SDS running buffer (Life Technologies), proteins were transferred to nitrocellulose membranes and blocked for 1 h in Tris-buffered saline with 0.1% Tween-20 (TBS-Tween) containing 5% skim milk with constant shaking. Membranes were then incubated with primary antibodies specific for CD51 (Bioss), Integrin beta 1 (Abcam), Integrin beta 3 (Cell Signaling), Integrin beta 5 (Cell Signaling) and Integrin beta 8 (Sigma-Aldrich) diluted in 5% BSA/TBS-Tween overnight at 4° C. with constant shaking. Membranes were then washed with TBS-Tween and incubated for 1 h at room temperature (RT) with horseradish peroxidase labelled secondary antibodies diluted in 5% skim milk or BSA/TBS-Tween for 1 h. Membranes were then washed again with TBS-Tween and then incubated for 5 min SuperSignal™ M West Pico PLUS Chemiluminescent Substrate (Life Technologies) before imaging with the Chemidoc® XRS+ camera (Biorad) and the Image Lab™ software.

### Pharmacological Inhibition of Pw1⁺ Cells In Vitro

FACS sorted PW1⁺ or PW1⁻ cells were plated at the density of 25 000 cell per cm² in culture plates coated with attachment factor (ThermoFisher Scientific). Cells were left to adhere overnight with 0.5 mL of growth medium (DMEM 10% FBS 1% P/S). The culture medium was changed the day after. At day 5, culture media was changed and supplemented with 0, 300 nM, or 1 μM of cilengitide. At day 6, medium was removed, and cells were washed twice with PBS. Cells were then lysed and RNA extracted followed by an RT-PCR. A Q-PCR was finally performed on selected fibrosis markers.

### Statistical Analysis

The data of this manuscript were expressed as mean±SEM. When comparing more than 2 groups, quantitative data were analyzed using one-way ANOVA and pair wise comparisons with Tukey's test for multiple comparisons. The Mann Whitney U test was used for comparing continuous variables between 2 groups. A log rank (Mantel-Cox) test was used to compare survival between groups. P-values <0.05 were considered significant.

### Results

### Bioinformatic Analysis Defines Cardiac PW1⁺ Cells Putative Membranome

We first characterized the transcriptome profile of cardiac PW1⁺ cells by RNA-sequencing. For this purpose, cardiac PW1⁺ were FACS-isolated from fresh cardiac tissues using our PW1^{nLacZ} reporter mouse model using C₁₂FDG, a fluorescent substrate for β-galactosidase activity. We obtained cardiac PW1⁺ from normal hearts as well as from ischemic hearts (i.e., seven days after MI). We then used filtered, aligned, quality-controlled RNA-seq output files to predict amino acid sequences of the corresponding genes and we then used a series of bioinformatics algorithms to identify putative membrane proteins (data not shown). Briefly, we considered proteins with a predicted N-terminal endoplasmic reticulum targeting signal peptide and predicted transmembrane domains but with no intracellular localization signals (i.e., no ER retention signal motif and no mitochondrial targeting peptide nor Nuclear export signal). By progressive filtering, we thus generated a list of 2040 candidate expressed in cardiac PW1⁺ cell membranes in both normal and ischemic conditions (data not shown). The characteristics of these 2040 candidates were further screened in available databases to identify 913 candidates as more likely expressed in the plasma membrane of cardiac PW1⁺ cells (data not shown). We then performed the same strategy to define the putative surface membranome of other cell types (i.e., cardiomyocytes, non-myocytes heart fractions and embryonic stem cells) and performed a last screen to limit our list to 378 candidates that were only observed in the datasets obtained in cardiac PW1⁺ cells (data not shown). Functional enrichment analysis of this short list of cardiac PW1⁺-specific and condition-insensitive cell surface markers identified an important number of transmembrane receptors, transporters, and a smaller proportion of ion channels and enzymes (data not shown). However, the molecular function was not described for the majority of the identified candidates.

### Proteomic Analysis Cross-Validates the Expression of 9 Cell Surface Proteins in Cardiac PW1⁺ Cells

In parallel of the transcriptomics approach, we also performed a proteomic analysis of FACS-isolated cardiac PW1⁺ cells using mass spectrometry (data not shown). We identified the expression of 1885 proteins which were further analyzed in available databases in order to restrict the dataset to 230 proteins with plasma membrane location. We then made a direct comparison of the transcriptomics and proteomics datasets and confirmed the cross-identification of 9 candidates with both approaches (data not shown). Among these 9 cell surface proteins, we found 3 transporters, 4 receptors and 2 enzymes and most of these candidates were involved in cell motility, adhesion to the matrix and response to wounding (data not shown). Five of these candidates were already described in non-cardiac cells as plasma membrane clusters of differentiation (i.e., CD51-Itgav, CD140a-Pdgfra, CD172a-Sirpa, CD39-Entpd1 and CD163) and were further considered for the remainder of our study.

### Itgav (CD51) is Highly Expressed in Cardiac PW1⁺ Cells

The expression of these 5 newly identified cell surface markers was analyzed by cytometry in C₁₂FDG⁺ cells isolated from PW1^{nLacZ} reporter mouse hearts. We strikingly observed that 92.98±1.01% of cardiac PW1⁺ cells express CD51 (data not shown). The four other new markers as well as typical adult stem cell markers (i.e., CD44, CD34, CD166) were expressed in a lower proportion (i.e., around 50%) of cardiac PW1⁺ cells (data not shown), thus indicating CD51 as a potent marker to discriminate cardiac PW1⁺ cells. Further analyses in sorted CD45− Ter119− cardiac cells showed the high clustering of FDG and CD51 expression, further confirming that resident cardiac PW1⁺ cells display a high expression level of CD51 (data not shown). Reciprocally, CD51 expression was observed in the majority of FDG⁺ CD45⁺ Ter119⁻ cardiac cells (data not shown).

In order to develop and validate a new sorting strategy for cardiac PW1⁺ cells based on CD51 expression, we first analyzed the expression of the four other studied candidates in CD51 sorted cells from PW1^{nLacZ}-reporter mice hearts and found that FDG⁺ CD51⁺ CD45⁻ cells exclusively express CD140a (i.e, PDGF receptor alpha) while FDG⁺ CD51⁺ CD45⁺ cells express CD172a (data not shown). Using our PW1^{nLacZ} reporter mouse, we then started cell selection using CD51, CD45, CD140a and CD172 in order to propose a new sorting strategy of cardiac PW1+cells. Briefly, the cardiac CD51⁺ populations can be easily segregated according to CD45, CD140a and CD172 expression (data not shown). We then assessed beta-galactosidase activity in each of these sub-populations and found that the new strategy allows to recover 84.57±1.61% of PW1+ cells among the CD51+ CD45− CD140a+ CD172− population and 91.13±0.81% among the CD51+ CD45+ CD140a− CD172+ population. These results indicated CD51 as being expressed in almost all cardiac PW1+cells, and as being predominantly found in cells expressing PW1⁺ in the myocardium.

### Cardiac PW1⁺ Cells Express Different αV-Containing Integrins

CD51 (Itgav) belongs to the family of integrins that are transmembrane receptors which act as bridges for cell-ECM connections and cell-cell interactions. We thus first analyzed CD51 expression in cardiomyocytes and non-myocytes fractions freshly isolated from mouse normal hearts. Huvec cells were used as positive controls. We found that CD51 was exclusively expressed in the non-myocytes fractions (data not shown). We then FACS-sorted PW1⁺ and PW1 cell fractions from normal and ischemic mice hearts and found that CD51 expression was only observed in cardiac PW1⁺ cells (data not shown), thus further corroborating the transcriptomics, proteomics and cytometry data. Western blots analyses further confirmed a significant increase in CD51 expression in ischemic hearts and more specifically in the infarct zone (data not shown), which is consistent with a predominant expression in cardiac PW1⁺ cells as we previously showed a significant increase in the number of cardiac PW1⁺ cells post-MI with a predominant location to the infarct area¹⁹.

Integrin alphaV subunits can combine to beta subunits to form different integrin combinations that can be more abundantly observed in some particular cell types. We thus sought for a peculiar integrin signature in cardiac PW1⁺ cells. We firstly assessed the expression of Itgav and beta subunits (including Itgb1, Itgb3, Itgb5, and Itgb8) using our transcriptomic data sets. In line with our western blot data, Itgav was predicted as more abundant in cardiac PW1⁺ cells than in other studied cell types. We then found that the mRNA expression level of Itgb1 was higher as compared to other integrin beta subunits in cardiac PW1⁺ cells as well as in other studied cell types (data not shown), suggesting a predominant αVβ1 complex in cardiac PW1⁺ cells. Western blotting in cardiac cell fractions (data not shown) confirmed the presence of ITGβ1 ITGβ3, ITGβ5, and ITGβ8 in non-myocytes cells. The presence of ITGβ1 but also of ITGβ3 was further confirmed in cardiac PW1⁺ cells while ITGβ5, and ITGβ8 were not observed in these cells (data not shown). Consistently, ITGβ1 and ITGβ3 were over-expressed in the infarct areas of post-MI hearts (data not shown). Overall, these results confirm the predominant expression of CD51 (i.e., ITGAV) in PW1⁺ cells in the myocardium and suggest the principal presence of αVβ1 and αVβ3 heterodimers in cardiac PW1⁺ cells.

### Targeting PW1^{+ α}V integrin Reduce Cardiac Fibrosis After MI

Recent evidence have shown αV integrins as central mediators of organ fibrosis through TGF-beta activation^{22,23} with a key role of αVβ1 integrin among others. Our results showed that CD51 can be used as a cell surface marker to identify PW1⁺ cells but we then wondered whether CD51 can also directly participate in the cardiac PW1⁺ fibrogenic behavior as previously described¹⁹. CD51 (αV integrin) recognizes and binds the tripeptide sequence RGD (arginine, glycine, aspartic acid) which is a key motif for cell-cell and cell-ECM interactions. Based on this motif, a cyclic RGD pentapeptide was developed (called cilengitide) and was shown to exert strong inhibiting activity against the integrin αV, including αVβ1, αVβ3 and αVβ5.

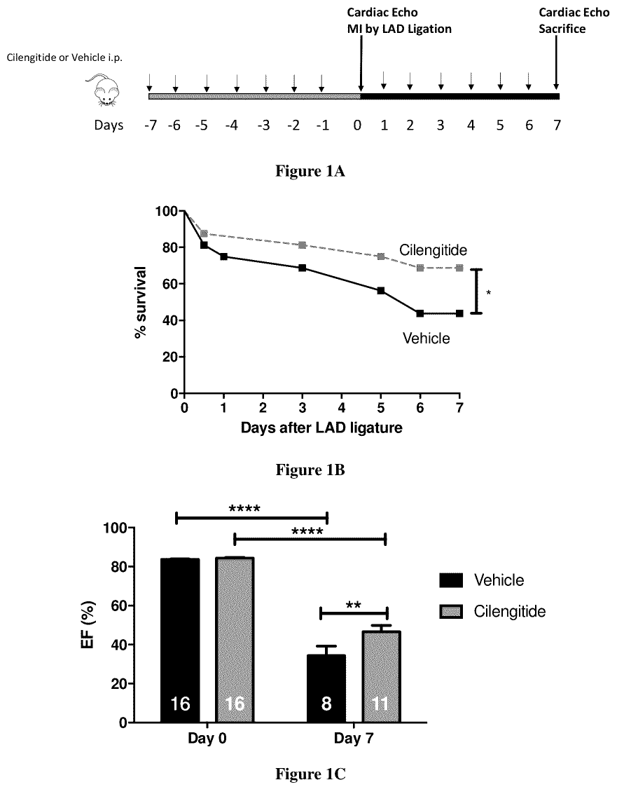

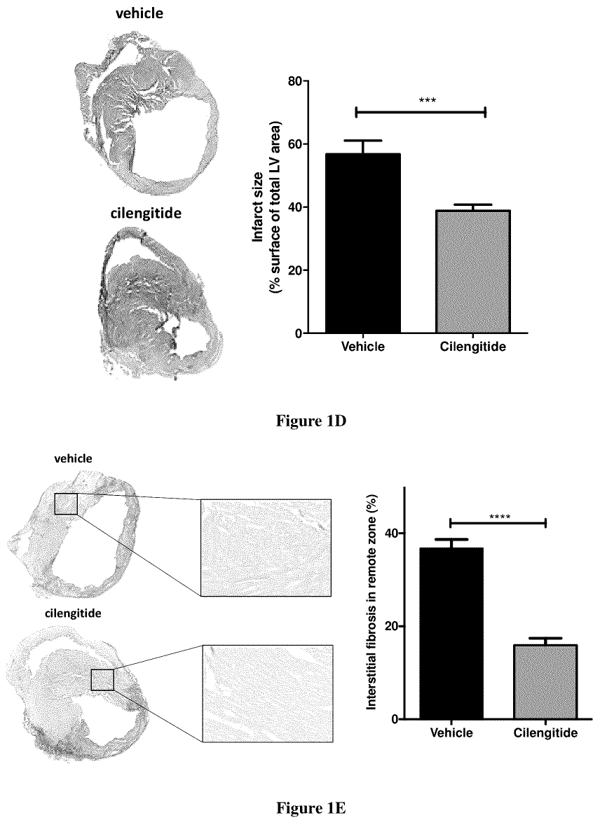

To further assess the potential contribution of αV integrin as found in cardiac PW1⁺ cells, we examined the potential of αV integrins pharmacological blockade to prevent cardiac fibrosis in response to an ischemic injury. Mice were pre-treated for 7 days with cilengitide or vehicle and were then subjected to MI via a complete LAD ligation. The mice were further daily treated with cilengitide or vehicle for additional 7 days before final evaluation of cardiac function and remodeling (FIG. 1a). We found that cilengitide treatment was associated 7 days after MI with a significant improvement in survival (FIG. 1b) that correlated with a significant increase in cardiac function as measured by left ventricular ejection fraction (FIG. 1c). Collagen (masson trichrome and picrosirius red) stainings were then performed on 8 sequential cardiac sections (data not shown). Digital image quantification demonstrated a significant reduction in infarct size (38.8±7.9% vs. 56.8±14.9% in cilengitide vs. vehicle respectively, p=0.0008, FIG. 1d) as well as in interstitial fibrosis measured in the remote myocardial area (15.9±10.4% vs. 36.8±13.1% in cilengitide vs. vehicle respectively, p<0.0001, FIG. 1e), while cilengitide treatment was not associated with any changes in survival, cardiac function or cardiac fibrosis in sham animals (data not shown).

### αV Integrin Blockade Reduces the Fibrogenic Behavior or Cardiac PW1⁺ Cells

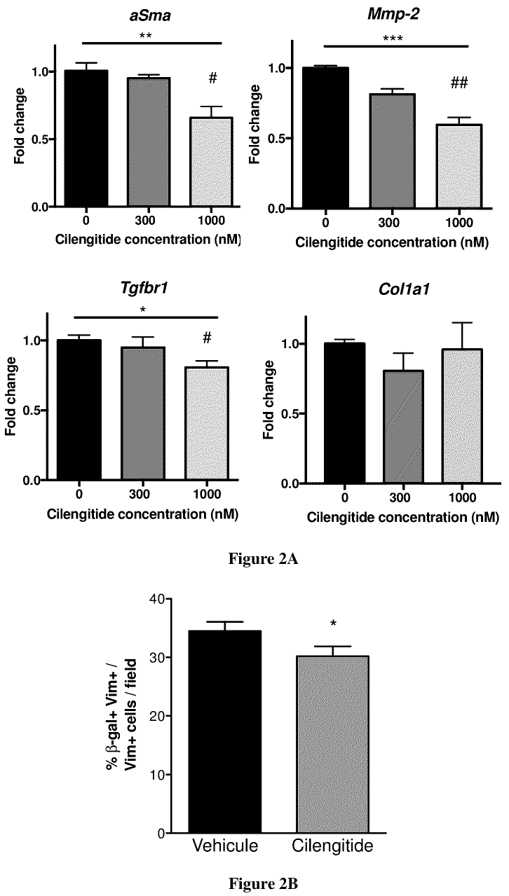

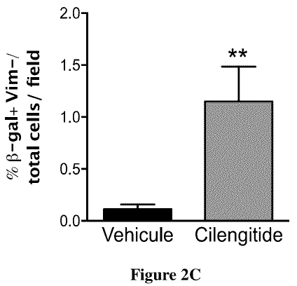

Overall, these results suggest a primary blunting of mechanisms leading to the development of cardiac fibrosis in response to MI, a phenomenon that could involve cardiac PW1⁺ cells as they were previously shown to display a fibrogenic behavior post-MI¹⁹ and we here showed that they predominantly express αV integrins which are targeted by cilengitide. To test this hypothesis, we first investigated whether CD51 pharmacological inhibition affects the expression of profibrotic genes in freshly FACS-isolated cardiac PW1⁺ cells. These cells were seeded and cultured for 4 days and then treated for 48 h with growing concentrations of cilengitide or vehicle. Cilengitide treatment resulted in significant down-regulation of aSma (or Acta2), mmp2 and tgfrb1 (FIG. 2a), suggesting a reduction in TGF-beta signaling and myofibroblast differentiation capacity. Colla1 expression was however not affected by cilengitide treatment, supporting a specific regulatory role on individual ECM genes.

We then performed short-term lineage tracing of cardiac PW1⁺ cells using PW1^{nLacz}, reporter mouse model. The stability (produrance) of the β-gal reporter indeed allows for identification of cells derived from β-gal⁺ cells. We examined β-gal activity and a typical fibroblast marker (i.e., vimentin) expression in vivo in cardiac sections of post-MI (7 days) hearts and found that cilengitide treatment was associated with a significant reduction in the proportion of cells co-expressing β-gal and vimentin (FIG. 2b). Reciprocally, we found a significant increase in the number of Δ-gal+cells that did not express vimentin after cilengitide treatment (FIG. 2c). Overall, these results suggest that αV integrin blockade with cilengitide impacted the ability of cardiac PW1⁺ cells to differentiate into fibroblast and contribute to fibrotic scarring.

### Discussion

To the best of our knowledge, we provide here the first description of proteins expressed at the cell surface of PW1⁺ cardiac adult stem cells. The identification of a cell-surface markers signature is critical for the isolation, characterization and understanding of resident adult stem cells as these cells typically represent a small proportion of the living cells in an organ. In addition, there is a diversity of markers that have been proposed but with a significant overlap between markers and a limited discriminatory power. For instance, we here found that typical stem cells markers (e.g., CD140a, CD44, CD166) are indeed found at the surface of cardiac PW1⁺ cells but in only ˜50% of these cells. So far, the identification of PW1⁺ cells has been made possible through the use of a transgenic PW1-reporter mouse model that revealed extremely potent to demonstrate PW1 as a pan-tissue stem cell marker^{19,20,27-29}. We here used this model to specifically isolate cardiac PW1⁺ cells and then performed a multi-omics unbiased approach to further decipher the cell-surface membranome of these cells. The transcriptomics predictions helped us to strikingly filter down the large number of membrane proteins identified through a mass spectrometry technique and therefore increased the likelihood to identify a more specific cell surface marker among the limited number of remaining candidates. A similar approach was recently proposed to define the secretome of bone marrow stem cells³⁰.

We then found CD51 as being expressed in the very large majority of cardiac PW1⁺ cells, a result that was not previously reported nor anticipated. Reciprocally, CD51 is predominantly found in cells expressing PW1⁺ in the myocardium thus highlighting the discriminative value of this new marker that could now serve for PW1⁺ cells sorting. Whether CD51 specifically tags PW1⁺ cells in non-cardiac organs however remains to be determined.

The identification of this new cell surface marker then provided important information on the pathophysiological role of cardiac PW1⁺ cells. CD51 is an αV-integrin subunit, a subset of integrin family cell adhesion receptors that were recently suggested as central mediators of organ fibrosis through TGF-beta activation^{22,23}. Strikingly, we recently reported that cardiac PW1⁺ cells display a fibrogenic behavior in response to ischemic injury, notably by directly giving rise to fibroblasts¹⁹. Our new results indicate that CD51 (or ITGAV) play a direct role in controlling the fibrogenic fate of cardiac PW1⁺ cells. We indeed found that blockade of αV-integrin limits the expression of fibrotic genes in isolated cardiac PW1+cells and reduce the ability of cardiac PW1⁺ cells to differentiate into fibroblasts in vivo in in a MI murine model. Pharmacological blockade of αV-integrin with cilengitide in this model significantly reduced the post-MI fibrotic remodeling, with a significant reduction in infarct size and interstitial fibrosis. The survival and cardiac function of cilengitide treated animals were consequently improved. As some integrins are expressed in endothelial cells, it has been suggested that cilengitide has some anti-angiogenic effects₃₁. However, this mechanism could not explain our current observations as an anti-angiogenic effect should have adversely deteriorated cardiac function post-MI. Similarly, ITGβ3 can be expressed by endothelial cells but we found a significant increase in ITGβ3 expression in the infarct area, a zone typically depleted in vessels but enriched with PW1⁺ cells. Our data rather show that αV-integrins are predominantly expressed in PW1⁺ cells within the cardiac cells, thus suggesting that the observed benefit on cardiac fibrotic remodeling is primarily carried out by reducing the fibrogenic behavior of these cells. A contribution of circulating cells (such as inflammatory cells in response to cardiomyocyte necrosis) can however not be ruled out.

The role of integrins in non-cardiac organ fibrosis (i.e., lung and liver) is becoming increasingly recognized and genetic deletion or pharmacological inhibition of integrins have been associated with reduced fibrotic remodeling^{23,32,33}. Interestingly, these recent studies indicate a critical role of αVβ1 in organ fibrosis^{23,32}. The role of αVβ1 heterodimers as well as mesenchymal-like cells αV integrins in the development of muscular and cardiac fibrosis have only been recently acknowledged^{23,34}. Our present data further provide evidence for a role of cardiac PW1⁺ adult stem cells in the development of cardiac fibrosis through αV integrins. Our results are compatible with the hypothesis that cardiac PW1⁺ cells represent a cellular source of fibrosis by directly differentiating into fibroblasts. It is however also possible that cardiac PW1⁺ cells indirectly contribute to fibrosis by orchestrating a microenvironment that further favors the activation of resident fibroblasts or the remodeling of the extracellular matrix. Our data suggest some specific changes in individual ECM genes (notably related to TGF-beta pathway) after αV-integrin blockade, an observation that will deserve further experiments.

Lastly, our study indicates that a new anti-fibrotic therapy could be developed by targeting αV-integrins as expressed in the fibrogenic cardiac PW1⁺ cells. So far, heart failure therapies are used to alleviate cardiac workload and indirectly reduce cardiac remodeling but there is currently no effective anti-fibrotic therapeutic strategy that could complement the current therapies for HF^{9,11,12}. To achieve an antifibrotic therapy, a better understanding of the mechanisms contributing to cardiac fibrosis is needed and our data report on a new cellular source of cardiac fibrosis. Importantly, in our study, pharmacological blockade of αV-integrin was associated with favorable outcomes in a murine MI model. Anti-fibrotic strategies in MI often exposed to the risk of cardiac rupture by limiting the development of replacement fibrosis which restores the region devoid of viable cardiomyocytes and prevents cardiac rupture³⁵. For instance, it was recently shown that strategies that limits inflammatory cells recruitment to the site of infarction delayed the removal of dead cardiomyocytes and their replacement by a scar tissue, therefore increasing susceptibility to cardiac rupture³⁶. We did not observe such an adverse outcome but rather found a significant improvement in survival under cilengitide treatment. Combined with the significant reduction in interstitial fibrosis in cilentigitide treated animals, this suggests that blockade of αV-integrin does not directly affect the replacement fibrotic remodeling but rather limits the development of reactive interstitial fibrosis in the viable myocardium¹².

Overall, these data identify cardiac PW1⁺ cells as a source of cardiac fibrosis in response to an ischemic injury via αV-integrin and suggest that pharmacological targeting of αV-integrin may provide clinical benefit in the treatment of cardiac ischemia.

### EXAMPLE 2

### Summary

Integrins are surface receptors that bind to extracellular matrix ligands and regulate cellular function through mechanical stress-initiated signal transduction. Integrin alpha-V (or CD51) is implicated in myocardial fibrosis and anti-CD51 therapy improves cardiac function and cardiac fibrotic remodeling post-myocardial infarction. However, their contribution in non-ischemic pressure-overload induced heart failure has not been established. We implanted male C57BL/6J wild-type mice with osmotic minipumps containing the vasoconstrictor Angiotensin II (AngII) (1.44 mg/kg/day), a combination of AngII and the al adrenergic agonist Phenylephrine (PE)(50 mg/kg/day), or saline as a control. Animals were treated with daily IP injections of the anti-CD51 molecule cilengitide or vehicle. Cardiac echography, flow cytometry, histological, protein analyses were used to study the development of fibrosis, adverse remodeling, cellular and molecular variations in the different mouse models of chemically-induced pressure-overload and in response to anti-CD51 therapy. Mice treated with the combination of AngII and PE showed a maladaptive cardiac hypertrophic remodeling with a rapid transition to heart failure and had an increased number of CD51+ cells. In contrast, mice treated with AngII alone had compensated cardiac hypertrophy with low levels of fibrosis and no signs of congestive heart failure and had no changes in CD51 expression. Anti-CD51 therapy in mice receiving AngII+PE significantly reduced the transition to heart failure and the development of cardiac fibrosis. In this model, anti-CD51 therapy reduced the recruitment of monocyte-derived pro-inflammatory CCR2+ macrophages, which also showed a high expression of CD51 at their surface. We finally found that macrophages sense matrix stiffness and activate a pro-inflammatory response to stiffer substrates, a response that was blunted by anti-CD51 therapy. Anti-CD51 therapy reduces the transition to heart failure in response to pressure overload and modulates the pro-inflammatory and deleterious action of CD51+ myeloid cells. We identified CD51 inhibition as a novel therapeutic strategy for reducing the progression of non-ischemic and pressure dependent heart failure.

### Methods

### Mouse Models

C57B1/6J male mice of 9 to 11 weeks old were purchased from Janvier Labs. Osmotic pumps were implanted subcutaneously in anesthetized mice back via inter-scapula incision. Two main protocols were used in which mice were separated in 4 groups. The first model is based on the delivery of Angiotensin II (1.44 mg/kg/day) for 28 days. The second model is based on the combined delivery of Angiotensin II (1.44 mg/kg/day) and Phenylephrine (50 mg/kg/day). A final group with single Phenylephrine delivery (50 mg/kg/day) was used to validate the synergic effect of dual Angiontensin II+Phenylephrine stimulation. Delivery of phosphate buffer saline was used as a control. Mice were then treated with daily IP injection with either RGD-cyclic molecule cilengitide (10 mg/kg/day) or vehicle, until sacrifice.

### Cardiac Echography

Cardiac echography was performed on anesthetized mice using a Vevo 2100 high resolution ultrasound device (Visualsonics, Toronto, Canada) with a 40 MHz probe (MS-550). Mice were anesthetized with 3% isoflurane in air for induction and maintained with 1.5%. Mice were depilated in the thoracic region and then placed in supine position on a dedicated heating platform, allowing monitoring of ECG, temperature and respiratory frequency. 3 Time Motion acquisition from Parasternal long axis views were recorded. Left Ventricle dimensions (internal diameter, septum and posterior wall thickness) were measured using the VevoLab Software (Visualsonics) that provided associated calculations (Fractional Shortening, Ejection Fraction, Cardiac Output). Mitral, aortic and pulmonary flows were recorded using PW Doppler mode providing velocities parameters (peak and mean velocities)

### Cell Isolation and Cytometry

Cells were isolated from the whole left ventricle following enzyme digestion. Briefly, mice were euthanized and the heart was excised. The atrium and the right ventricle were cut out and the left ventricle was rinsed out of its blood. The left ventricle was then cut into small pieces (0.5-1 mm³) using scalpel and surgical scissors and directly placed into 2 mL of enzymatic solution: Collagenase II-500U/mL (Worthington-WOLS04177), DNase I-1,6 U/mL (Roche, 11284932001), in HBSS with Mg²⁺Ca²⁺for 15 minutes at 37° C. The tissue was then quickly homogenized using trituration with 1000 μL pipette. Supernatant was extracted and put in STOP solution: HBSS with Mg2+Ca2++10% FBS ((Hyclone FBS (U.S.), GE Healthcare Life Sciences, SH30071.03), on ice. Pellet was filled with new enzymatic solution for another 15 min and the process was repeated until complete digestion. Cells homogenate was then filtered on a 70 μm strainer and stained with selected antibodies before being passed in the BD LSRFortessa™ X-20 Cell Analyzer.

### Histological Analysis

Fibrosis assessment was performed using picrosirius red staining on either 4 μm FFPE cuts or 8 μm cryosections. Images were taken using Hamamatsu scanner at 20× and analysis was performed using both in-house ImageJ macro or FIBER-ML program developed in our center.

Cardiomyocyte size and capillary density were assessed using WGA (FL-1021, vectorlabs) to tag cardiomyocyte plasma membrane and CD31 (DIA-310, dianova) to tag endothelial cells. Images were taken using confocal microscopy (Leica SPE) at 40× and analyzed using in-house ImageJ macro.

### In Vitro BMDM Differentiation

Briefly, bone marrow was extracted from 11 weeks old C57B16/J mice femur and sterilized by a brief dip in ethanol and sterile Phosphate Buffered Saline (Gibco™M PBS) then sectioned in half. Bone marrow was collected by centrifugation (10,000 g for 30 sec at 4° C.), then cells were filtered using a 70 μm strainer and resuspended in growth medium (Gibco™ Milieu RPMI 1640, 10% FBS, 1% Penicillin Streptomycin, 5.000 U/mL). To stimulate macrophage differentiation, the growth media was supplemented with recombinant mouse Macrophage Colony Stimulating Factor (M-CSF, 100 ng/ml). Cell density was determined with a cell counter and the cells were seeded into T75 for 7 days at 37° C. in 5% CO₂ at a density of 1×10⁶ cells/cm².

### Differential Matrix Stiffness Macrophages Culture

Briefly, after 7 days of differentiation, MO macrophages were trypsinized and reseeded at a density of 1.2×10⁶ cells/well on P6 wells with either 8 kPa or 16 kPa hydrogel bottom, coated with vitronectin (0.5 μg/cm²). Macrophages were let 5 hours to adhere to the matrix, then treated with cilengitide (10 μM) or vehicle (PBS) for 24 hours. Supernatant was then collected, aliquoted and snap frozen for further analysis.

### Immunoflex Assay

Supernatant was used undiluted with Bio-Rad Bio-Plex Pro Mouse Cytokine 23-plex Assay #M60009RDPD kit, according to the manufacturer protocol. The plate was analyzed on a Bio-Plex 200 system.

### Statistical Analysis

Data sets from the models comparison were analyzed using a One-way ANOVA with multiple comparison, followed by a Tukey post-hoc test. The data sets from the HHF model with cilengitide treatment and in vitro experiments were analyzed using a 2-way ANOVA with multiple comparison followed by a Tukey post-hoc test. p<0.05 was considered statistically significant. The data are presented as Mean±SD. GraphPad Prism 9 software (GraphPad Software, San Diego, CA) was used for the statistical analyses.

### Results

Models of Adaptive vs. Maladaptive Cardiac Hypertrophy

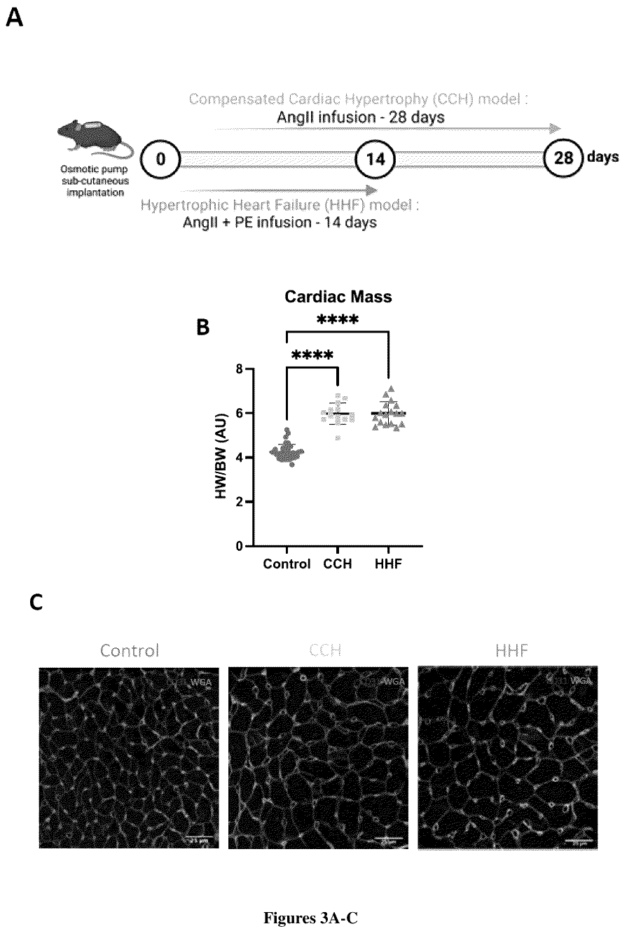

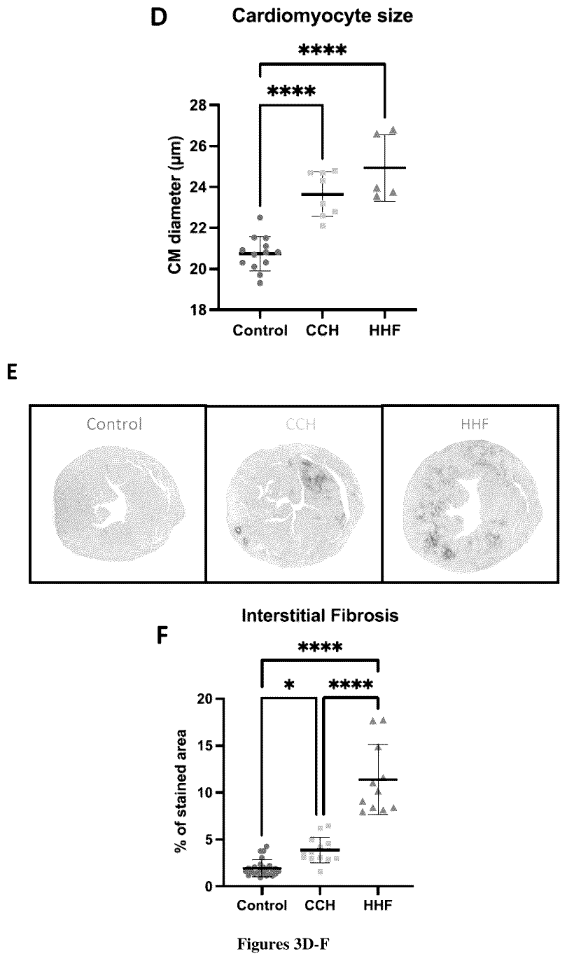

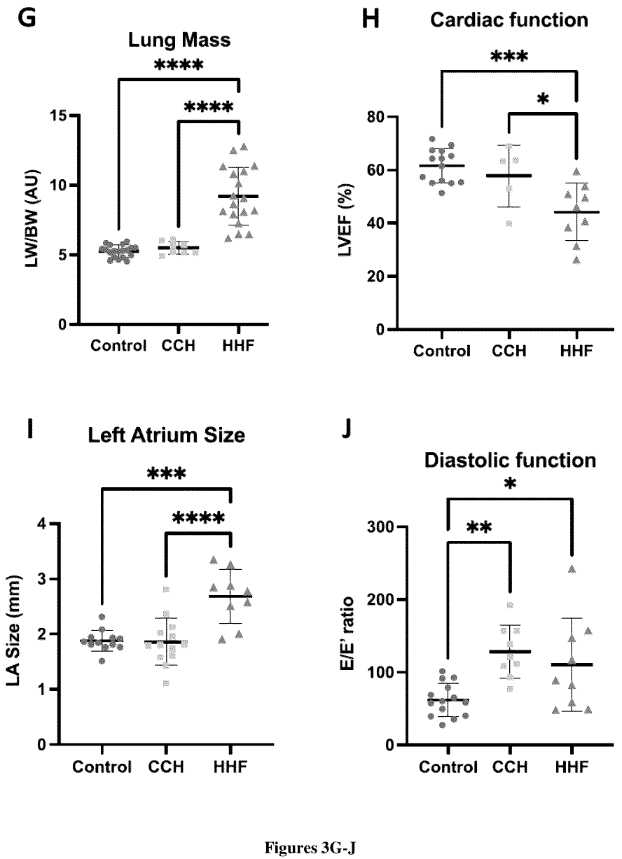

We evaluated the development of cardiac hypertrophy, fibrosis and dysfunction in different mouse models of chemically-induced pressure-overload. Male C57BL/6J wild-type mice were either implanted with osmotic minipumps containing the vasoconstrictor Angiotensin II (AngII) (1.44 mg/kg/day), a combination of AngII and the al adrenergic agonist Phenylephrine (PE)(50 mg/kg/day), or phosphate buffer saline as a control (FIG. 3A). The duration of administration was planned for 28 days but was reduced to 14 days in the group receiving the dual stimulation because of a high mortality and a significant transition to heart failure observed as early as Day 14 (FIG. 3A).

We identified the mouse model of single AngII stimulation as a compensated cardiac hypertrophy (CCH) model and the model with dual stimulation (AngII+PE) as a hypertrophic heart failure (HHF) model. Indeed, we found that the hypertrophic remodeling was comparable between models either at the level of the organ (FIG. 3B) or at the cellular level as shown with cardiomyocytes size measurements (FIG. 3C-D). However, despite a similar cardiac hypertrophic remodeling and increase in blood pressure, we found significant differences in the development of interstitial fibrosis between models. Using picrosirius red staining to stain the collagen fibers, we found that the HHF model led to a significantly higher level of fibrosis as compared to the levels observed in the CCH model (8.7%±3.7 vs. 3.9%±1.4, p<0.001, FIG. 3E-F). The level of interstitial fibrosis was significantly higher in the CCH model as compared to control, however with levels that were globally in the supra-physiological range and below 5%. We also found that mice treated with PE alone minorly increased cardiac mass without significant increase in interstitial fibrosis, thus identifying a synergic effect of AngII+PE on the development of cardiac fibrosis.

On top of the fibrotic remodeling, dual stimulation with AngII+PE was associated with a rapid development of congestive signs in the HHF animals with a significant increase in lung congestion (FIG. 3G). Importantly, none of the mice in the CCH model developed a significant lung congestion while having similar levels of cardiac hypertrophy (FIG. 3B, G). Single PE stimulation also did not change the lung mass. Echocardiographic analysis also showed a borderline reduction in systolic function in HHF mice, as well as a significant increase in the left atrium size (FIG. 3I). Here again, these abnormalities were not found in CCH mice or in mice treated with PE alone. However, both CCH and HHF mice had a modified E/E′ ratio (FIG. 3J). Capillary density was increased in both CCH and HHF groups. Together, these results indicate that both CCH and HHF mice display a cardiac hypertrophic remodeling with relaxation trouble, but that the functional consequences and deterioration to congestive heart failure is only observed in HHF mice.

### CD51 Expression is Increased in the Hypertrophic Heart Failure Model

We further investigated the mechanisms supporting the differences in fibrosis development and the transition to heart failure between these two models. We have previously shown that cardiac stromal cells that express integrin αV (CD51) are involved in the formation of fibrotic scar in a mouse MI model with permanent LAD ligation REF JACC.

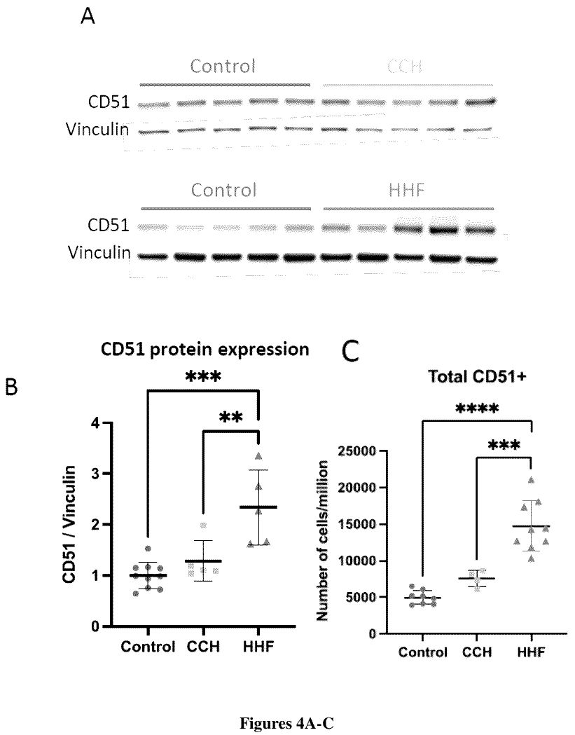

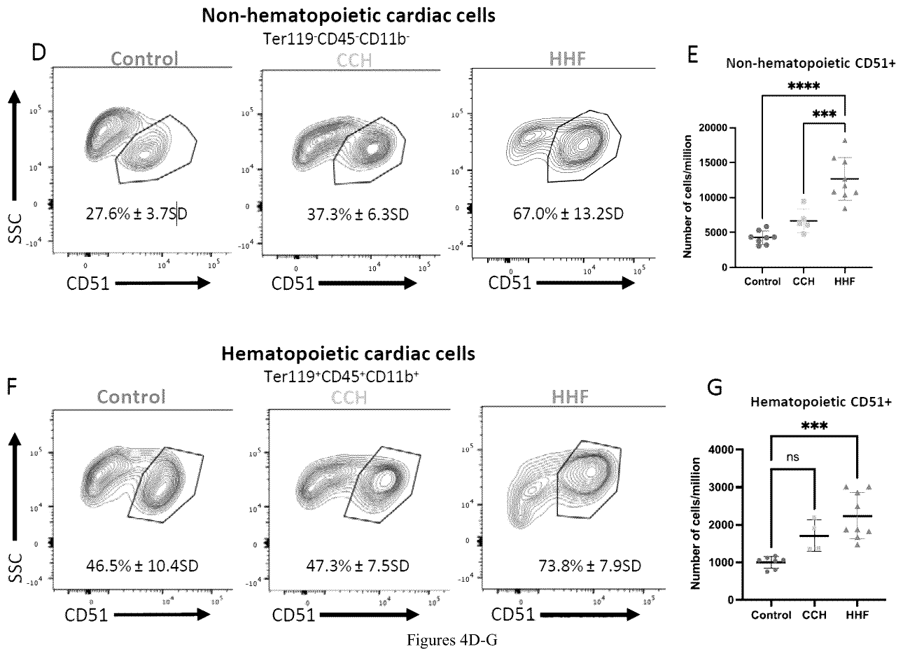

Pharmacological blockade of integrin αV (CD51) with the RGD-binding molecule cilengitide significantly reduced deleterious fibrotic remodeling and also cardiac dysfunction post-MI REF Sci Rep. We thus interrogated CD51 expression in our cardiac hypertrophy models and found a significant increase in HHF mice but not CCH mice, as seen by western-blotting (FIG. 4A,B) on isolated left ventricles (LV). We also isolated non-cardiomyocytes cells from LV and found a significantly higher number of CD51+ cells in the HHF group as seen by flow cytometry analysis (FIG. 4C). In line with the limited level of fibrotic remodeling, the number of CD51+ cells was not increased in the CCH group. Using different markers, we then separated non-hematopoietic cardiac cells from hematopoietic cardiac cells in LVs from animals in the HHF group. Interestingly, we found that the increase of CD51 expression in the HHF model was observed on both non-hematopoietic (FIG. 4D-E) and hematopoietic cardiac cells (FIG. 4F-G). These results expand our previous observations post-MI by identifying an increased expression of CD51 in a different mouse model that combines both a fibrotic remodeling and a rapid transition to heart failure.

### The Anti-Integrin CD51 Cilengitide Reduces Fibrosis and the Transition to HF

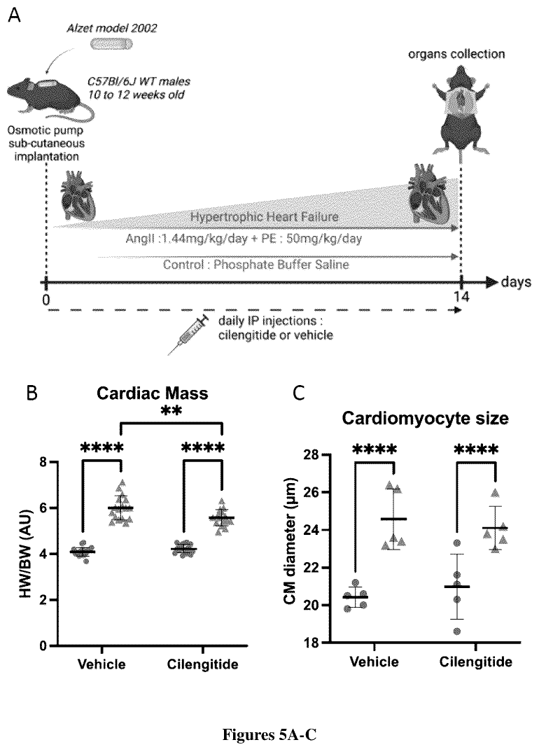

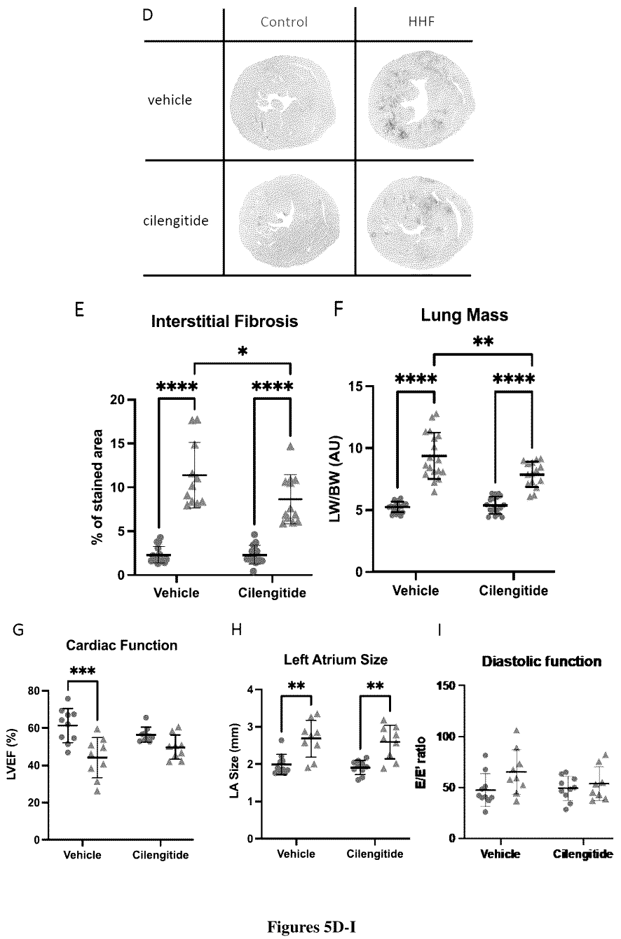

We next tested the preventive effect of CD51 pharmacological inhibition in both our models. HHF mice were treated with daily IP administrations of cilengitide or vehicle for 14 days (FIG. 5A). We first observed that the cardiac mass was slightly but significantly reduced in response to cilengitide at the organ level, as assessed both by organ weighting (FIG. 5B) and echocardiography. However, the size of cardiomyocytes remained unchanged (FIG. 5C), indicating that this effect is not explained by a simple reduction in hypertrophic remodeling but rather by a reduction in the content of the interstitial compartment. Cilengitide was associated with a significant reduction in the development of interstitial fibrosis as identified by picrosirius red staining (10.2%±4.4 vs. 7.6%±1.8, p<0.05, FIG. 3D,E). No difference in perivascular fibrosis was found. In addition, cilengitide significantly reduced the transition to heart failure as indicated by a significant reduction in lung mass (FIG. 5F) and a preservation of LVEF (FIG. 5G). Markers of diastolic dysfunction as well as left atrium enlargement were unchanged or tended to be improved (FIG. 5 H,I).

In contrast to the HHF model, cilengitide administered for 28 days by daily IP injection in the CCH model was not associated with any major difference in the development of interstitial fibrosis, cardiac hypertrophy or cardiomyocyte size. Cilengitide did not change capillary density or blood pressure in this model, consistent with a lack of direct vascular impact.

Taken together, these results show that CD51 pharmacological blockade has a preventive impact on the development of both cardiac fibrosis and heart failure in the context of cardiac hypertrophic remodeling induced by pressure overload.

### CD51 Identifies Inflammatory Monocytes and Monocyte-Derived Macrophages

Concordant with our previous description in the mouse post-MI model, CD51+ non-hematopoietic cells were found to express typical markers of cardiac stromal cells including

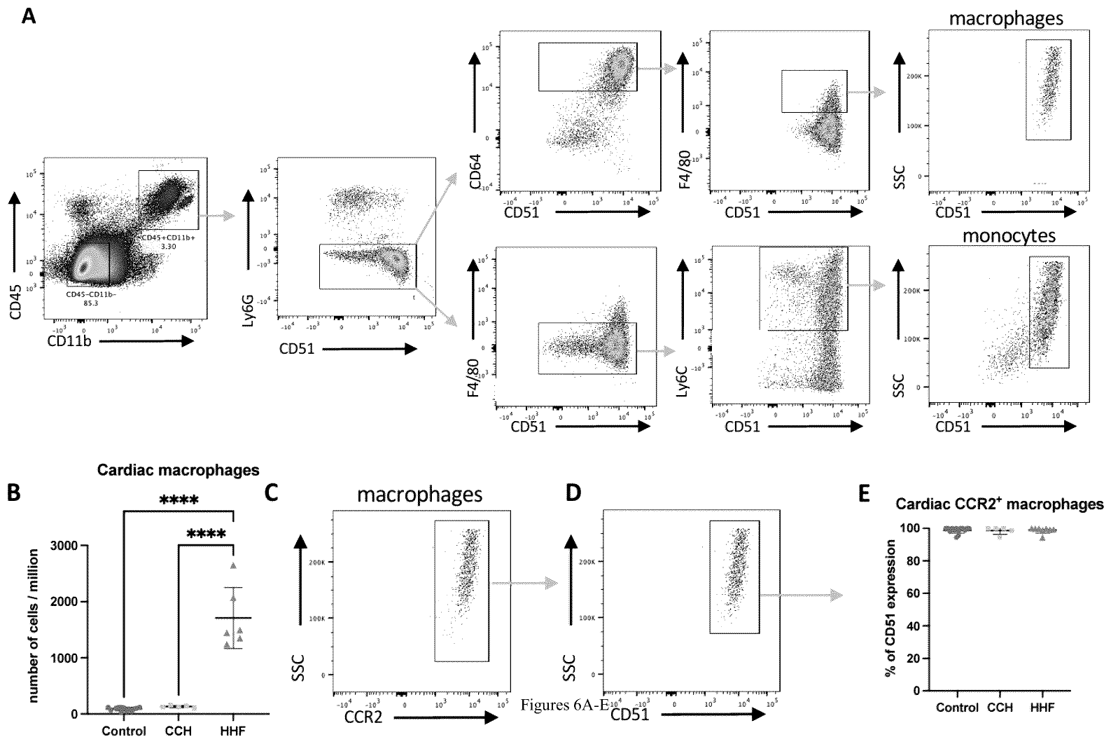

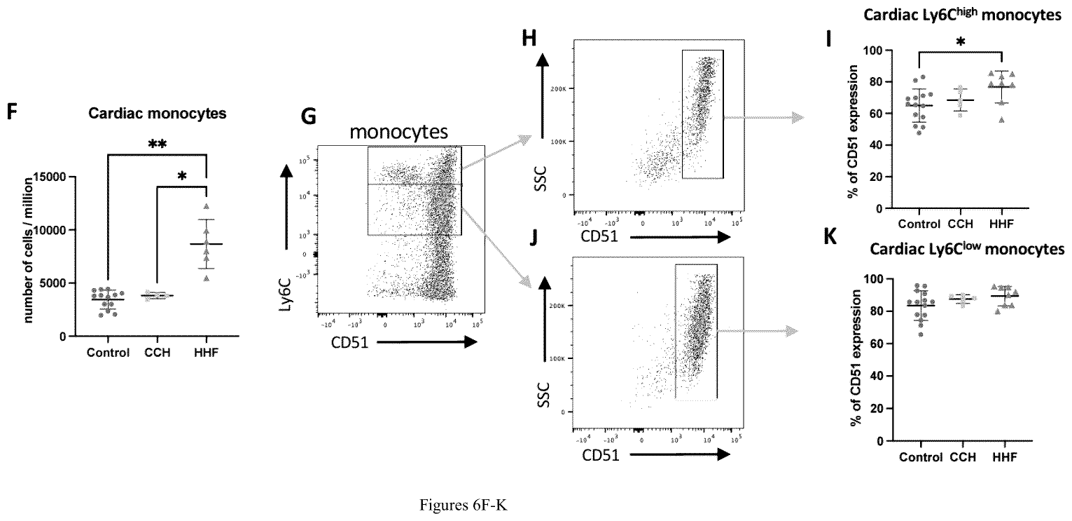

PGDFR-alpha and PW1 and here again, the number of those cells was found to be increase in the HHF but not CCH model. We also performed deeper flow cytometry analysis of CD51+ cells expressing markers of the hematopoietic compartment (FIG. 4F, G), and identified two populations of cells with a high percentage of CD51 expression, namely the monocytes and the macrophages (FIG. 6A). Both monocyte and macrophage numbers were increased in HHF but not CCH mice, confirming the differential involvement of these myeloid cells between these two models (FIG. 6B, F). We further investigated the exact nature of the monocytes and macrophages present in the heart of HHF mice to find that a vast majority of the macrophages were CCR2-positive (FIG. 6C), thus mostly corresponding to monocyte-derived macrophages with a pro-inflammatory phenotype. Amongst those cells, almost 100% expressed CD51 (FIG. 6D-E).

Finally, we separated cardiac monocytes according Ly6C expression levels (mid and high) and found that 70% of the Ly6C^{hi} monocytes and more than 80% of ly6C^{mid} monocyte express CD51 (FIG. 6I, K). Interestingly, the high percentage of CD51 expression was particularly observed in monocytes isolated from the cardiac tissue as compared to other tissue such as the spleen. Together these results indicate that HHF mice recruit monocyte-derived pro-inflammatory CCR2+ macrophages, with both cell type showing a high expression of CD51, making them targets for anti-integrin therapy such as cilengitide.

### Cilengitide Reduces the Inflammatory Response in HHF Animals

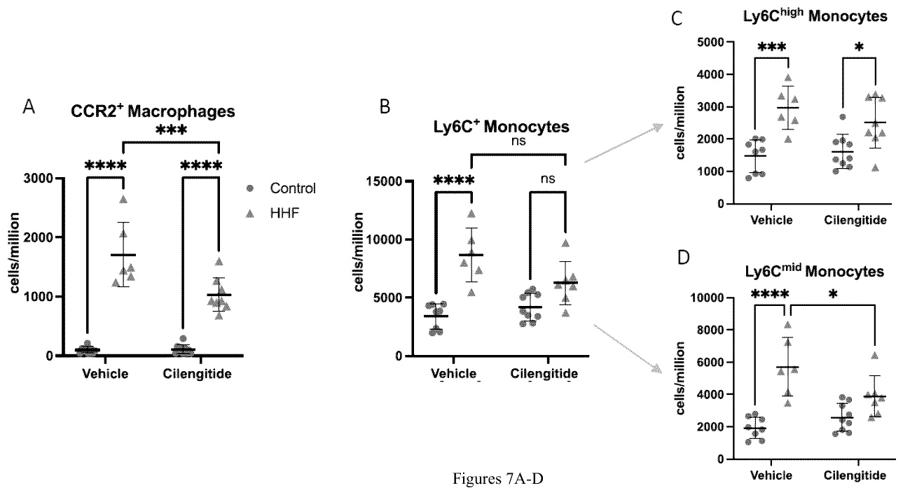

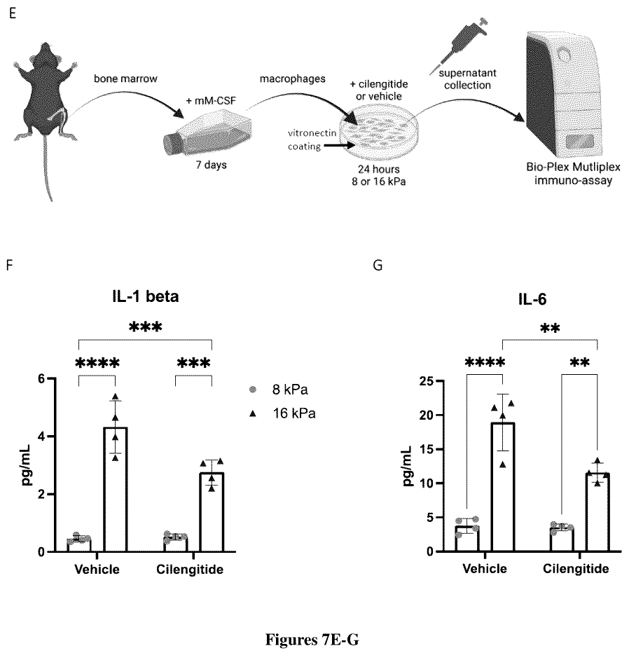

Using cytometry analysis, we analyzed changes in the number of CD51+ cardiac macrophages and monocytes after cilengitide blockade in HHF mice. Cilengitide significantly reduced the number of CCR2⁺ macrophages as well as Ly6C^{mid} but not Ly6C^{high} monocytes number (FIG. 7A-D), suggesting a direct effect of the integrin blockade in the recruitment of pro-inflammatory monocytes (and latter macrophages) into the myocardial tissue. Of note, cilengitide did not modify the amount of Ly6C⁺ monocytes in the blood stream, bone marrow or spleen, indicating a cardiac-specific effect in the HHF condition.

### CD51 Blockade Blunts the Inflammatory Reaction of Cardiac Myeloid Cells

CD51 is an integrin that mediate adherence to extracellular matrix components, notably vitronectin. We thus interrogated whether CD51 blockade disrupts the interaction of CD51+ immune cells with the ECM and blunts the sensing of a stiffer fibrotic ECM and their consequent cytokines' secretion. We therefore differentiated bone marrow cells from 12 weeks old mice for 7 days, into naive MO macrophages. We then cultivated those macrophages on vitronectin matrix with two different stiffness (i.e. 8 kPa and 16 kPa), in order to mimic stiffening conditions found in a healthy vs. a fibrotic myocardium respectively (FIG. 7E). After letting the macrophages to adhere, we cultivated them for 24 hours with cilengitide or vehicle. The cytokines secretion profile was analyzed by collecting the supernatant and analyzing its composition using an Immunoflex assay recognizing 23 different analytes. In line with our hypothesis, we found a significant increase in the expression of the pro-inflammatory cytokines in response to a stiffer ECM. Among these factors, the increase in IL-1 beta and IL-6 was however significantly reduced by almost 40% after cilengitide treatment (FIG. 7F,G), suggesting a modulation of the secretion of pro-inflammatory factors by targeting CD51.

### Discussion

Our findings underscore an important role of cells expressing CD51 (i.e., integrin alphaV) in the maladaptive response to cardiac pressure overload. Our results show a significant increase in the expression of CD51, as well as a significant increase in the number of CD51+ cells, in animals presenting a rapid transition to HF following chemically-induced pressure overload (HHF with dual stimulation AngII+PE) but not in animals with an adaptive cardiac hypertrophic response (CCH with single AngII infusion). This result was observed despite identical levels of cardiac hypertrophic remodeling in both models, thus suggesting a mechanism independent of cardiomyocytes-specific signaling. We found that CD51 is highly expressed by cardiac stromal cells expressing typical markers such as PDGFRa or PW1. These cells have been shown to contribute to cardiac fibrosis by favoring myofibroblasts formation or activating pro-fibrotic pathways related to TGF-beta. Concordantly, a significant increase in the level of cardiac fibrosis was found in the HHF group. However, the absolute levels were too limited (around 10% of total area) to fully explain the rapid transition to HF in these animals, prompting us to investigate other components related to CD51+. Recently, a functional role for monocyte-derived macrophages has been established in the transition from compensated hypertrophy to heart failure (HF) (Patel JACC BTS and Liao×PNAS 2018) and we concordantly found a high expression of CD51 in monocytes and macrophages isolated from the cardiac tissues of HHF mice. Notably, the increase in CD51 expression was not observed in myeloid cells from the blood, the spleen or the bone marrow, suggesting a specific increase in the diseased organ. In addition, CD51+ macrophages express C-C Chemokine Receptor type 2. CCR2⁺ macrophages that are thought to derive from infiltrating monocytes and were recently shown to regulate myocardial inflammation and heart failure pathogenesis. Recruited macrophages have also been implicated in the development of cardiac fibrosis in a CCR2-dependent process. The use of CCR2 expression to distinguish recruited from resident cardiac macrophages has been recently challenged as CCR2+ resident cardiac macrophages from hematopoictic origins has been recently identified (BajPai Cric Res 2019). However, we found that almost 100% of CCR2+ macrophages expressed CD51, suggesting that this surface marker can tag both populations. Other subsets of resident macrophages have been described but were marginally observed in our models.

The reduction in heart failure transition in response to a pharmacological blockade of CD51 is another important finding of our study. We have previously shown that cilengitide significantly ameliorates cardiac outcome after MI in murine model, through the targeting of cardiac stromal CD51+ cells and reduced their differentiation in myofibroblasts. Our results show that the development of cardiac fibrosis was slightly but significantly decreased in our HHF model, thus strengthening evidence that targeting integrins can represent an appealing anti-fibrotic strategy. Similarly, integrin alphaV blockade has been associated with reduced liver and pulmonary fibrosis. Our results now show that CD51 pharmacological blockade could also be used to limit the transition from compensated to decompensated cardiac hypertrophy, which is a novel finding. Here, we propose that a part of this preventive effect relates to a modulation of the pro-inflammatory and deleterious action of CD51+ myeloid cells. It has been suggested that matric stiffening can regulate macrophage polarization and exacerbate their pro-inflammatory responses, but whether this process is observed in the cardiac tissue is under-explored PMID: 34292877, PMID: 29522370. Recent reports have shown the presence of mechanically-gated sensors in immune cells, their ability to transduce a mechanical stimulus to these cells and the consequent activation of a pro-inflammatory response PMID: 35645593. Concordantly, our results show that macrophages display a pro-inflammatory secretion profile in response to a stiffer substrate (i.e. 16 KPa) that was selected to mimic the mechanical conditions of a myocardial tissue with fibrotic remodeling. Our findings show that the sensing of changes in the mechanical constraints is partly conveyed by integrins that are typically known to mediate adherence to extracellular matrix components. In addition to its known effect on fibroblast activation, we report here that the targeting of CD51/Integrin alpha V results in a significant reduction in the number of infiltrating CCR2+ macrophages and we suggest that it also results in a significant decrease in their secretion of major pro-inflammatory cytokines. Our results add to the growing interest for targeting integrins in cardiac disorders by showing a novel immunomodulation impact. Inflammation is a hallmark of heart failure. However, the therapeutic immunomodulation in HF requires a better understanding of the respective role of the different immune cells. Previous studies have reported the expansion of CCR2+ monocytes /macrophages during the early phases of cardiac remodeling following pressure overload induced by a thoracic aortic banding. Concordant with our results, specific and circumscribed inhibition of CCR2⁺ monocytes and macrophages early during pressure overload reduced pathological hypertrophy, fibrosis, and systolic dysfunction during the late phase of pressure overload. Antibody-mediated depletive strategies against CCR2 have been proposed, but a strong limitation of macrophage depletion strategies is the targeting of myeloid populations in other tissues (such as the spleen or the lung) and the related side effects. Similarly, selective CCR2 antagonists can have systemic impact that will limit its translation to the clinics. Importantly, we found that CD51 was highly expressed by activated cardiac monocytes/macrophages but to a much lower level in other organs, thus suggesting CD51 pharmacological blockade as a more specific way to target CCR2+ cardiac macrophages in the hypertrophic hearts.

Macrophages can communicate and interact with multiple cells, including fibroblasts. Resident and recruited monocytes/macrophages have a direct role in regulating fibrosis, however with contrasted effects according to macrophages subsets. A recent study has reported that resident cardiac macrophages prevent fibrosis in a pressure overload model, whereas recruited monocyte-derived macrophages stimulate fibrosis. In our study, CD51 blockade was associated with a significant reduction of fibrosis formation, but this effect can also be explained by the targeting of CD51 expressed by cardiac stromal cells which have a fibrogenic potential as previously shown. Fibroblasts can also sense matrix stiffness and mechanotransduction signaling pathways can activate their pro-fibrotic program PMID: 28534817.

In conclusion, we report on the critical role of CD51-expressing cells in the process of maladaptive cardiac remodeling in response to pressure overload. CD51 expression identifies different cell subsets, including CCR2+ monocytes/macrophages. Pharmacological inhibition of CD51 reduces the transition to heart failure, and modulates the pro-inflammatory and deleterious action of CD51+ myeloid cells. We identified CD51 inhibition as a novel therapeutic strategy for reducing the progression of non-ischemic and pressure dependent heart failure.

### REFERENCES

Throughout this application, various references describe the state of the art to which this invention pertains. The disclosures of these references are hereby incorporated by reference into the present disclosure.
1. Maggioni, A. P., et al. EURObservational Research Programme: regional differences and 1-year follow-up results of the Heart Failure Pilot Survey (ESC-HF Pilot). Eur J Heart Fail 15, 808-817 (2013).
2. Roger, V. L., et al. Heart disease and stroke statistics—2012 update: a report from the American Heart Association. Circulation 125, e2-e220 (2012).
3. Seferovic, P. M., et al. Organization of heart failure management in European Society of Cardiology member countries: survey of the Heart Failure Association of the European Society of Cardiology in collaboration with the Heart Failure National Societies/Working Groups. Eur J Heart Fail 15, 947-959 (2013).
4. Ponikowski, P., et al. 2016 ESC Guidelines for the diagnosis and treatment of acute and chronic heart failure: The Task Force for the diagnosis and treatment of acute and chronic heart failure of the European Society of Cardiology (ESC)Developed with the special contribution of the Heart Failure Association (HFA) of the ESC. Eur Heart J 37, 2129-2200 (2016).
5. Savarese, G. & Lund, L. H. Global Public Health Burden of Heart Failure. Card Fail Rev 3, 7-11 (2017).
6. Dunlay, S. M., Roger, V. L. & Redfield, M. M. Epidemiology of heart failure with preserved ejection fraction. Nat Rev Cardiol 14, 591-602 (2017).
7. Lewis, G. A., et al. Biological Phenotypes of Heart Failure With Preserved Ejection Fraction. J Am Coll Cardiol 70, 2186-2200 (2017).
8. Gyongyosi, M., et al. Myocardial fibrosis: biomedical research from bench to bedside. Eur J Heart Fail 19, 177-191 (2017).
9. Schelbert, E. B., Fonarow, G. C., Bonow, R. O., Butler, J. & Gheorghiade, M. Therapeutic targets in heart failure: refocusing on the myocardial interstitium. J Am Coll Cardiol 63, 2188-2198 (2014).
10. Li, A. H., Liu, P .P., Villarreal, F. J. & Garcia, R. A. Dynamic changes in myocardial matrix and relevance to disease: translational perspectives. Circ Res 114, 916-927 (2014).
11. Gourdie, R. G., Dimmeler, S. & Kohl, P. Novel therapeutic strategies targeting fibroblasts and fibrosis in heart disease. Nat Rev Drug Discov 15, 620-638 (2016).
12. Travers, J. G., Kamal, F. A., Robbins, J., Yutzey, K. E. & Blaxall, B. C. Cardiac Fibrosis: The Fibroblast Awakens. Circ Res 118, 1021-1040 (2016).
13. Kramann, R., et al. Perivascular Gli1+ progenitors are key contributors to injury-induced organ fibrosis. Cell Stem Cell 16, 51-66 (2015).
14. Kalluri, R. The biology and function of fibroblasts in cancer. Nat Rev Cancer 16, 582-598 (2016).
15. Teekakirikul, P., et al. Cardiac fibrosis in mice with hypertrophic cardiomyopathy is mediated by non-myocyte proliferation and requires Tgf-beta. J Clin Invest 120, 3520-3529 (2010).
16. Olivey, H. E., Mundell, N. A., Austin, A. F. & Barnett, J. V. Transforming growth factor-beta stimulates epithelial-mesenchymal transformation in the proepicardium. Dev Dyn 235, 50-59 (2006).
17. Duan, J., et al. Wnt1/betacatenin injury response activates the epicardium and cardiac fibroblasts to promote cardiac repair. EMBO J 31, 429-442 (2012).
18. Humphreys, B. D., et al. Fate tracing reveals the pericyte and not epithelial origin of myofibroblasts in kidney fibrosis. Am J Pathol 176, 85-97 (2010).
19. Yaniz-Galende, E., et al. Fibrogenic Potential of PW1/Peg3 Expressing Cardiac Stem Cells. J Am Coll Cardiol 70, 728-741 (2017).
20. Besson, V., et al. PW1 gene/paternally expressed gene 3 (PW1/Peg3) identifies multiple adult stem and progenitor cell populations. Proc Natl Acad Sci U S A 108, 11470-11475 (2011).
21. Mitchell, K. J., et al. Identification and characterization of a non-satellite cell muscle resident progenitor during postnatal development. Nat Cell Biol 12, 257-266 (2010).
22. Henderson, N. C., et al. Targeting of alphav integrin identifies a core molecular pathway that regulates fibrosis in several organs. Nat Med 19, 1617-1624 (2013).
23. Chen, C., Li, R., Ross, R. S. & Manso, A. M. Integrins and integrin-related proteins in cardiac fibrosis. J Mol Cell Cardiol 93, 162-174 (2016).
24. Chen, C., Khaleel, S. S., Huang, H. & Wu, C. H. Software for pre-processing Illumina next-generation sequencing short read sequences. Source Code Biol Med 9, 8 (2014).
25. Li, B. & Dewey, C. N. RSEM: accurate transcript quantification from RNA-Seq data with or without a reference genome. BMC Bioinformatics 12, 323 (2011).
26. Yates, A., et al. Ensembl 2016. Nucleic Acids Res 44, D710-716 (2016).
27. Dierick, F., et al. Resident PW1+ Progenitor Cells Participate in Vascular Remodeling During Pulmonary Arterial Hypertension. Circ Res 118, 822-833 (2016).
28. Sojoodi, M., et al. The zinc finger transcription factor PW1/PEG3 restrains murine beta cell cycling. Diabetologia 59, 1474-1479 (2016).
29. Besson, V., et al. Expression Analysis of the Stem Cell Marker Pw1/Peg3 Reveals a CD34 Negative Progenitor Population in the Hair Follicle. Stem Cells 35, 1015-1027 (2017).
30. Korf-Klingebiel, M., et al. Myeloid-derived growth factor (C19orf10) mediates cardiac repair following myocardial infarction. Nat Med 21, 140-149 (2015).
31. Nisato, R. E., Tille, J. C., Jonczyk, A., Goodman, S. L. & Pepper, M. S. alphav beta 3 and alphav beta 5 integrin antagonists inhibit angiogenesis in vitro. Angiogenesis 6, 105-119 (2003).
32. Reed, N. I., et al. The alphavbetal integrin plays a critical in vivo role in tissue fibrosis. Sci Transl Med 7, 288ra279 (2015).
33. Conroy, K. P., Kitto, L. J. & Henderson, N. C. alphav integrins: key regulators of tissue fibrosis. Cell Tissue Res 365, 511-519 (2016).
34. Murray, I .R., et al. alphav integrins on mesenchymal cells regulate skeletal and cardiac muscle fibrosis. Nat Commun 8, 1118 (2017).
35. Sun, Y. & Weber, K. T. Infarct scar: a dynamic tissue. Cardiovasc Res 46, 250-256 (2000).
36. Lorchner, H., et al. Myocardial healing requires Reg3beta-dependent accumulation of macrophages in the ischemic heart. Nat Med 21, 353-362 (2015).

1. A method of treating cardiac fibrosis in a patient in need thereof comprising administering to the patient a therapeutically effective amount of a αV-integrin inhibitor.

2. The method of claim 1, wherein cardiac fibrosis arises from aging, exposure to certain drugs, or in response to heart disease or hypertension.

3. The method of claim 2, wherein the heart disease is myocardial infarction.

4. The method of claim 2, wherein the heart disease is non-ischemic pressure-overload induced heart failure.

5. The method of claim 1, wherein the αV-integrin inhibitor limits the development of reactive interstitial fibrosis in the viable myocardium.

6. The method of claim 1, wherein the αV-integrin inhibitor improves cardiac function.

7. The method of claim 1, wherein the αV-integrin inhibitor is an antibody.

8. The method of claim 7, wherein the antibody is specific for αV-integrin.

9. The method of claim 1, wherein the αV-integrin inhibitor is Cilengitide.

10. The method of claim 1, wherein the V-integrin inhibitor is an inhibitor of αV-integrin.


SEQUENCE LISTING (1 sequence)

SEQ ID NO: 1 - protein, 1048 aa, Homo sapiens
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHVFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVPVWVIILAVLAGLLLLAVLVFVMYRMGFFKRVRPPQEEQEREQLQPHENGEGNSET